(19) United States
(12) Patent Application Publication
(10) Pub. No.: US 2025/0058298 A1
(43) Pub. Date: Feb. 20, 2025

(54) SEPARATION MATRIX AND METHODS FOR SEPARATING TARGET MOLECULES

(71) Applicant: Cytiva BioProcess R&D AB, Uppsala (SE)

(72) Inventors: Jean-Luc Maloisel, Uppsala (SE); Åsa Hagner McWhirter, Uppsala (SE); Ola Lind, Uppsala (SE); Ida Helena Vestin, Uppsala (SE); Erik Tingborn, Uppsala (SE); Björn Norén, Uppsala (SE)

(21) Appl. No.: 18/712,580

(22) PCT Filed: Dec. 6, 2022
(86) PCT No.: PCT/EP2022/084530

§ 371 Date: May 22, 2024

(30) Foreign Application Priority Data
Dec. 7, 2021 (GB) 2117619.3

(51) Int. Cl. B01J 20/28; B01J 20/24; B01J 20/282; B01J 20/32

(52) U.S. Cl. CPC B01J 20/28078; B01J 20/24; B01J 20/28092; B01J 20/282; B01J 20/3219; B01J 20/3285; B01J 20/3293

(57) ABSTRACT

The present disclosure is directed to a separation matrix comprising a plurality of chromatography particles, each chromatography particle comprising a core and a layer surrounding the core, wherein the core has a first average pore diameter and the layer surrounding the core has a second average pore diameter, wherein the second average pore diameter is at least 1.5 times higher than the first average pore diameter, wherein the first average pore diameter excludes diffusion of a target molecule through the pores of the core and wherein the second average pore diameter at least partly permits diffusion of the target molecule through the pores of the layer surrounding the core. Further disclosed are a method for preparing such a separation matrix, uses of such a separation matrix and methods for separating target molecules by use of such a separation matrix, in particular a method for separating adeno associated virus capsids fully packaged with genetic material from adeno associated virus capsids not fully packaged with genetic material, and compositions obtained by said method.

### FIELD OF THE DISCLOSURE

The present disclosure relates to the field of separation of target molecules and is directed to a separation matrix comprising a plurality of chromatography particles, each chromatography particle comprising a core and a layer surrounding the core, designed such that a target molecule is excluded from diffusing through the pores of the core while the target molecule is permitted to diffuse at least partly through the pores of the layer surrounding the core.

Further disclosed are a method for preparing such a separation matrix, uses of such a separation matrix and methods for separating target molecules by use of such a separation matrix. In particular, a method for separating adeno-associated virus capsids fully packaged with genetic material from adeno-associated virus capsids not fully packaged with genetic material, and compositions, including pharmaceutical compositions, obtained by said method, as well as uses of such compositions, are disclosed.

### BACKGROUND OF THE DISCLOSURE

Peak broadening in chromatography is affected by different types of diffusion present in chromatography. Van Deemter has described three different terms that affect peak broadening in chromatography, the A, B and C terms (see e.g., T. Edge or P. J. Marriott). The A-term is the so called “Eddy diffusion”, and zone broadening is related to channeling through a non-ideal packing. The B-term is the longitudinal diffusion, one example of longitudinal diffusion is the diffusion of the analyte in the capillaries of a chromatography system. Longitudinal diffusion is more applicable for low molecular weight molecules since they have much higher diffusion rates in liquids than large proteins. It is important to have low dead volume in chromatography systems to reduce longitudinal diffusion. The C-term diffusion is caused by the mass transfer resistance between the mobile phase and the solid support. Diffusion occurs since the analyte diffuses in and out of the pores and the longer distance (larger particle size) the larger dilution of the analyte and furthermore peak broadening will appear. Increased flow rate also contributes to peak broadening since the diffusion rate into the pore is constant and an increased flow rate will separate the analyte further.

To optimise a process related to the purification of a specific target molecule, unique operating conditions will be required, and the best separation matrix will vary from case to case. Specific processes need to be designed for the purification of peptides and proteins, nucleic acids, virus particles etc. In the purification of antibodies, the type of antibody will be decisive for the choice of separation matrix. The same is true for different serotypes of adeno-associated viruses.

Adeno-associated viruses (AAV) are non-enveloped viruses that have linear single-stranded DNA (ssDNA) genome and that can be engineered to deliver DNA to target cells. Recombinant adeno-associated virus (rAAV) vectors have emerged as one of the most versatile and successful gene therapy delivery vehicles. There is an increasing demand to use viral vectors for gene therapy. The AAV vector is one of the most attractive gene transfer tools for developing novel genetic therapies for muscle diseases as well as other disorders. Most of the earlier AAV gene transfer studies used AAV serotype 2 (AAV2). To further improve the efficiency and specificity of AAV-mediated gene transfer, numerous AAV serotypes and variants have been developed by viral genome engineering and/or capsid modification. Use of serotypes like AAV8 and AAV9 have increased in recent years. Target organs determine selection of serotype. To use AAV particles as vectors in therapy it is necessary to purify the virus particles from cell impurities like DNA after transfection. Ultracentrifugation is efficient but not scalable. Normally, several filtration steps and several chromatography steps are used to separate AAV particles from cell cultures (see e.g., Weihong Qu et al).

Therapeutic efficacy of AAV vectors is dependent on high percentage of virus particles fully packaged with genetic material of interest. Upstream expression systems deliver a mixture of fully packaged AAV particles (containing the genetic material of interest), empty AAV particles, and AAV particles which are partially packaged with genetic material of interest), together with impurities. There is thus a need to enrich fully packaged AAV particles in the purification process. However, there are several challenges in relation to achieving an efficient and scalable separation of fully packaged and empty adeno-associated virus capsids, such as:
Large diversity of capsids (serotypes and variants) and cell culture differences in terms of yield of full capsids, which means that extensive optimization is needed for purification of each serotype or variant of adeno-associated virus.
Small differences between fully packaged and empty capsids in relation to several parameters relevant for purification, e.g., isoelectric point;
In addition to fully packaged and empty capsids, also partially packaged capsid variants are produced in the infected host cells. There are indications that such partially packaged, and thereby therapeutically less effective, capsids may be partly co-eluted with fully packaged capsids.

Consequently, there is still a need in this field of alternative separation matrices to provide novel chromatography materials and purification strategies to increase the speed, improve the resolution, and decrease the cost of the purification process and to provide large-scale methods for downstream processing of different types of target molecules.

### SUMMARY OF THE DISCLOSURE

The object of the present disclosure is to provide a chromatography material which is applicable in large-scale methods for separation of target molecules, and which achieves improved resolution in industrial preparative chromatography. The main application area for the presently disclosed chromatography material is the polishing step of a separation method, also called secondary or final purification.

More particularly, a first aspect of the present disclosure is directed to a separation matrix comprising a plurality of chromatography particles, each chromatography particle comprising a core and a layer surrounding the core,
wherein the core has a first average pore diameter and the layer surrounding the core has a second average pore diameter, wherein the second average pore diameter is at least 1.5 times higher than the first average pore diameter,
wherein the first average pore diameter excludes diffusion of a target molecule through the pores of the core and wherein the second average pore diameter at least partly permits diffusion of the target molecule through the pores of the layer surrounding the core.

A second aspect of the present disclosure relates to a method for preparing a separation matrix according to the first aspect, the method comprising preparing a plurality of chromatography particles.

A third aspect of the present disclosure is directed to use of the separation matrix according to the first aspect for preparative applications, such as for separating a target molecule from a cell culture harvest or impurities.

A fourth aspect of the present disclosure relates to a method of preparative chromatography for separating one or more target molecules from one or more other compounds in a liquid sample, which method comprises contacting a mobile phase comprising the target molecule(s) and compound(s) with the separation matrix according to the first aspect of the present disclosure.

A fifth aspect of the present disclosure is directed to a particular method of preparative chromatography for separating one or more target molecules from one or more other compounds in a liquid sample. More specifically, disclosed is a method for separating adeno-associated virus capsids fully packaged with genetic material from adeno-associated virus capsids not fully packaged with genetic material, the method comprising the following steps:
a) adding a liquid sample comprising adeno-associated virus capsids to a weak cation exchange chromatography material,
   wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
   wherein the weak cation exchange chromatography material comprises:
      i. a ligand for binding to the adeno-associated virus capsids; or
      ii. a support, which comprises the separation matrix according to the first aspect of the present disclosure, as described above;
b) eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

The above-disclosed method according to the fifth aspect may further comprise subjecting the eluate fractions comprising adeno-associated virus capsids fully packaged with genetic material, eluted in step (b), to one or more of the following steps:
c1) concentrating the adeno-associated virus capsids to a pharmaceutically relevant dose,
c2) replacing a buffer applied in step (b) of claim 1 with a pharmaceutically acceptable buffer, and/or
c3) sterilizing the eluate fractions comprising adeno-associated virus capsids, thereby obtaining a pharmaceutical composition comprising adeno-associated virus capsids.

A sixth aspect of the present disclosure is directed to a composition comprising adeno-associated virus capsids obtained by performing the above-described method according to the fifth aspect, in which composition the ratio of adeno-associated virus capsids fully packaged with genetic material to adeno-associated virus capsids not fully packaged with genetic material is at least 3:2.

Preferred aspects of the present disclosure are described below in the detailed description and in the dependent claims.

### BRIEF DESCRIPTION OF THE DRAWINGS

FIG. 1 schematically illustrates a chromatography particle comprising a core and a layer surrounding the core, which chromatography particle is comprised by a separation matrix according to the first aspect of the present disclosure.

FIG. 2 schematically illustrates a chromatography particle comprising a core, a first layer surrounding the core, and a second layer surrounding the first layer, which chromatography particle is comprised by a separation matrix according to the first aspect of the present disclosure.

FIG. 3a and FIG. 3b are flow charts of methods for preparing a separation matrix, comprising preparing a plurality of chromatography particles, according to the second aspect of the present disclosure.

FIG. 4 is a flow chart of a method for separating adeno-associated virus capsids according to the fifth aspect of the present disclosure.

FIG. 5 is a flow chart of the method for separating adeno-associated virus capsids according to FIG. 4, further comprising an additional step (al) before step (a).

FIG. 6 is a flow chart of the method for separating adeno-associated virus capsids according to FIG. 4, optionally comprising step (al) before step (a), further comprising one or more additional steps (c1), (c2) and/or (c3) after step (b).

FIG. 7 is a graph showing the elution curves for fully packaged and not fully packaged capsids according to separation methods as described in Example 1 below.

FIG. 8 is a graph showing the elution curves for monoclonal antibodies according to separation methods as described in Example 2 below.

FIG. 9 is a graph showing pore size distribution curves for chromatography particles according to an embodiment of the invention, obtained by loading polypeptides of a range of molecular weights on a chromatography column packed with the chromatography particles, and plotting the log Mw for the respective polypeptides against the gel phase distribution coefficient (Kₐᵥ).

### DETAILED DESCRIPTION OF THE DISCLOSURE

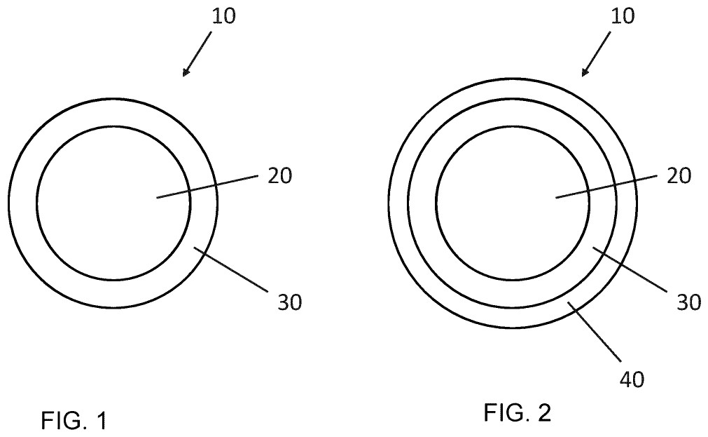

The present disclosure solves or at least mitigates the problems associated with peak broadening in chromatography in relation to existing chromatography media by providing, according to a first aspect of the present disclosure, a separation matrix comprising a plurality of chromatography particles (10). As schematically illustrated by FIG. 1, each chromatography particle (10) comprises a core (20) and a layer (30) surrounding the core (20), wherein the core (20) has a first average pore diameter and the layer (30) surrounding the core (20) has a second average pore diameter, wherein the second average pore diameter is at least 1.5 times higher than the first average pore diameter, wherein the first average pore diameter excludes diffusion of a target molecule through the pores of the core (20) and wherein the second average pore diameter at least partly permits diffusion of the target molecule through the pores of the layer (30) surrounding the core (20).

Since the core of the particle has an average pore diameter which does not permit diffusion of target molecules into and within the core, there will only be diffusion of target molecules in the layer surrounding the core (i.e., close to the surface of the particle). This results in shorter diffusion distances than when using an equally sized particle which has a homogeneous average pore diameter throughout its entire structure or volume.

A shorter diffusion distance leads to faster mass transfer of analytes in chromatography columns packed with the presently disclosed particles, which in turn results in sharper, narrower peaks of eluted analytes in the resulting chromatogram, i.e., an improved resolution of different analytes. The signal to noise ratio will benefit from sharper peaks. The resolution achieved by using the presently disclosed particles of a certain size is equal to the resolution achieved with smaller particles having homogeneous average pore diameter. However, the back pressure created in a chromatography column packed with the presently disclosed particles of a certain size will be equal to the back pressure created in a chromatography column packed with same-sized particles having a homogeneous average pore diameter. Thus, the back pressure will be lower for the presently disclosed particles of a certain size than for smaller particles, which might otherwise be chosen to obtain the required resolution of target molecules. This is an advantage of the presently disclosed particles compared to smaller particles, which cause higher back pressure and therewith associated limitations/problems.

The chromatography particle as disclosed herein may be said to comprise a porous core and a porous layer surrounding the porous core. As mentioned above, the core has a first average pore diameter and the layer surrounding the core has a second average pore diameter, wherein the second average pore diameter is at least 1.5 times higher, such as 1.5, 2, 2.5, 3, 3.5, 4, or 5 times higher, than the first average pore diameter. For example, the second average pore diameter may be at least 10 times, such as at least 15 times, such as about 20 times, higher than the first average pore diameter. The first average pore diameter excludes diffusion of a target molecule through the pores of the core, meaning that target molecules are excluded from diffusing into and through the porous structure of the core. Similarly, the second average pore diameter at least partly permits diffusion of the target molecule through the pores of the layer surrounding the core is intended to mean that target molecules are permitted to diffuse, completely or at least partly, into and through the porous structure of the layer surrounding the core.

It is to be understood that a chromatography particle, core, or layer, respectively, which is defined as having a certain “average pore diameter” or “average pore size” nevertheless may comprise pores of different sizes or diameters, both pores that are large enough to easily allow a target molecule to diffuse within the particle and pores that are small enough to exclude diffusion of the target molecule. This diversity of pore diameter or pore size can be measured by the diffusion coefficient of a molecule of a well-defined molecular weight and hydrodynamic size. As a non-limiting example, dextran having a molecular weight of 140-225 KDa, and a hydrodynamic diameter of 20-25 nm, can be used to evaluate the degree of diffusion of adeno-associated virus capsids within the pores of the core and the pores of the layer surrounding the core, respectively.

The term “target molecule” has its conventional meaning in the field of bioprocessing, in which target molecules are produced (often recombinantly) by cells in a cell culture and purified from the cell culture by any means of separation and purification. Alternatively, the target molecules are present in a biological solution which does not necessarily originate from a cell culture. Non-limiting examples of target molecules are biomacromolecules, which are large biological polymers that are made up of monomers linked together, e.g., peptides comprising at least 30 amino acid residues, proteins (which can be native or recombinant), including but not limited to enzymes, antibodies and antibody fragments, and further nucleic acid sequences, such as oligonucleotides, DNA and RNA (such as mRNA), as well as virus particles. A target molecule to be separated from other compounds by use of the presently disclosed chromatography particle is typically a virus particle, a protein or polypeptide, particularly a therapeutic protein or polypeptide, such as an antibody, or a virus particle comprising genetic material of interest (e.g., for therapeutic applications) but may also be, for example, a nucleic acid. A target molecule may for example be a biopharmaceutical, i.e., a biological molecule, including but not limited to a biological macromolecule, which is intended for use as a pharmaceutical compound. More particularly, non-limiting examples of target molecules contemplated herein include virus particles, virus-like particles, monoclonal antibodies (including but not limited to monoclonal antibody variants like bispecific, fusion, and chimeric monoclonal antibodies), and antibody fragments (including but not limited to antigen-binding fragments F(ab′)2, Fab, Fab′, and Fv). Non-limiting examples of virus particles are adeno-associated virus capsids, lentivirus capsids, adenovirus capsids, influenza virus capsids, and rotavirus capsids. Examples of adeno-associated virus capsids include capsids of different serotypes, such as adeno-associated virus serotype 9 (AAV9), adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), and adeno-associated virus serotype 10 (AAV10), and variants thereof. It is to be understood that “a target molecule” is intended to mean a type of target molecule and that the singular form of the term may encompass a large number of individual target molecules, or specimens, of the same type.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification. Herein, a virus particle to be separated from a cell culture by the presently disclosed method may alternatively be referred to as a “target molecule”, “target”, “analyte of interest”, or “analyte”. It is to be understood that “a virus particle” is intended to mean a type of virus particle and that the singular form of the term may encompass a large number of individual virus particles. Herein, the term “virus particle” may be used interchangeably with the terms “vector” and “capsid”, respectively, as further defined below.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses. Herein, the term “vector” may be used interchangeably with the terms “virus particle” and “capsid”, respectively.

The term “capsid” means the shell of a virus particle. The capsid surrounds the core of the virus particle, and normally should comprise a viral genome. A modified (recombinant) capsid, as produced in an upstream process of manufacturing, is supposed to comprise a complete viral genome, which genome includes genetic material of interest for one or more applications, for example of interest for various therapeutic applications. However, owing to low packaging efficiency, assembled capsids do not always contain any genetic material or only encapsidate truncated genetic fragments, resulting in so-called empty capsids and partially filled capsids, respectively. These capsids possess no therapeutic function, yet they compete for binding receptors during the cell-mediated processes. This may diminish the overall therapeutic efficacy and trigger undesirable immune responses. As a result, tracking these capsids throughout the production process is crucial to ensure consistent product quality and a proper dosing response (Xiaotong Fu et al). In up to 20-30% of a population of virus particles artificially produced in a cell culture, the capsid is only partially filled with genetic material. Further, in up to as much as 98% of artificially produced virus particles, the capsid does not comprise any part of the viral genome at all, i.e., it is empty. However, generally between 80% to 90% of artificially produced virus particles have empty capsids, and best cases currently achieve as little as 50% empty capsids.

Herein, the term “capsid” may be used interchangeably with the terms “vector” and “virus particle”, respectively. In the context of the present disclosure, a capsid may or may not comprise genetic material.

The term “genetic material of interest” is intended to mean genetic material which in the field of bioprocessing is considered relevant and valuable to get produced by viral replication and to purify such that it can be used in various applications, such as, but not limited to, therapeutic applications. As a non-limiting example, genetic material of interest may comprise a therapeutically relevant genetic material, such as a therapeutically relevant nucleotide sequence.

The term “capsid fully packaged with genetic material” is herein used to denote a capsid which has been correctly produced (by the host cell), or in other words,
a capsid which comprises a complete viral genome, or in other words,
a capsid comprising 100% of its viral genome, or in other words,
a capsid comprising a functional viral genome.

The viral genome includes a genetic insert, comprising genetic material of interest, as defined elsewhere herein.

A capsid which comprises a complete viral genome may herein alternatively be called a “full capsid” or a “fully packaged capsid”. The terms “full capsid”, “fully packaged capsid”, and “capsid fully packaged with genetic material” may be used interchangeably throughout this text.

The term “capsid not fully packaged with genetic material” is herein used to denote a capsid which has not been correctly produced (by the host cell), or in other words,
a capsid which does not comprise a complete viral genome, or in other words,
a capsid which comprises less than 100% of its viral genome.

A capsid which is not fully packaged with genetic material is either partially filled with genetic material or is not filled with any genetic material at all.

The term “capsid not fully packaged with genetic material” encompasses the terms “partially filled capsid” and “empty capsid”, as defined below.

A “partially filled capsid” is herein defined as a capsid which comprises parts of its viral genome, such as defective parts of its viral genome, or in other words,
a capsid which comprises a partial viral genome, or in other words,
a capsid which comprises a non-complete viral genome, or in other words,
a capsid which comprises a defective viral genome, or in other words,
a capsid which comprises more than 0% and less than 100% of the complete viral genome, such as from about 1% to about 99%, such as from about 5% to about 95%, such as from about 10% to about 90%, or such as about 1%, about 5%, about 10%, about 20%, about 30%, about 40%, about 50%, about 60%, about 70%, about 80%, about 90%, about 95%, or about 99%, of the complete viral genome. Since a partially filled capsid is an incorrectly produced capsid, it is desirable to separate and remove as many as possible of the partially filled capsids from a population of capsids, before putting the population of capsids to use in its intended application, e.g., a therapeutic application. Herein, a partially filled capsid may alternatively be called an “intermediate capsid”.

An “empty capsid” is herein defined as a capsid which does not comprise any part of its viral genome, i.e., which comprises 0% of its viral genome, or in other words,
a capsid which is not filled with any genetic material at all. Thus, an empty capsid does not comprise any genetic material of interest. Consequently, it is desirable (and sometimes required, e.g., due to clinical regulations) to separate and remove as many as possible of the empty capsids from a population of capsids, before putting the population of capsids to use in its intended application, e.g., a therapeutic application.

Before putting a population of virus particles to use in its intended application, e.g., a therapeutic application, it is desirable (sometimes even required, e.g., due to clinical regulations) to enrich the full capsids, i.e., to increase the percentage of full capsids at the expense of the percentage of partially filled capsids and empty capsids.

The percentage of full capsids and empty capsids in a population of capsids can be estimated or analyzed with several methods known in the art. Some of these methods are briefly described below:
1: A260:280 in chromatogram will give an estimation of percentage full capsids present in peaks (ratio 1-1.5 indicate enriched in full capsids, ratio 0.5-0.7 is containing mainly empty capsids).
2. qPCR:ELISA ratio. qPCR quantifies viral genomes and ELISA quantifies total viral particles. A ratio of 2 assays with variation is less accurate and will be uncertain. Requires orthogonal analysis for confirmation (see below, 3, 4 or 5).
3. Analytical anion exchange separating full and empty capsids (A260:280 ratio and peak area to calculate the percentage). Accuracy dependent of peak definition.
4. Analytical ultracentrifugation (AUC). Detects and quantifies particles of different density (corresponding to full, partially filled, and empty capsids). This is currently known as the “golden standard” in the art. However, ultracentrifugation is not scalable and thus is not suitable for analysis of large-scale batches of capsids.
5. Transmission electron microscopy (TEM). Image analysis counting particles (full, partially filled, and empty capsids). May introduce artifacts from sample preparation.

Some methods for estimating or analyzing the percentage of full capsids and empty capsids in a population of capsids are described in more detail in Xiaotong Fu et al, which is hereby incorporated by reference herein.

The term “variant” in relation to an adeno-associated virus serotype is intended to mean a modified or engineered adeno-associated virus serotype, in which the capsid structure has been modified to improve clinical performance, for example towards a specific target organ. As a non-limiting example, an adeno-associated virus serotype 9 (AAV9) variant comprises capsid parts of AAV9 and may additionally comprise capsid parts of other AAV serotypes than AAV9, such as AAV5. However, an AAV9 variant as referred to herein must retain a significant structural similarity to a non-modified AAV9 capsid, such as retaining at least 50%, such as 60%, 70%, 80%, or 90%, of the external surface structure of a non-modified AAV9 capsid. In the context of purification or separation of a variant of AAV9, a “variant” is herein defined as an adeno-associated virus which has a functionally equivalent binding capacity to the ligand of a specified chromatography material, compared to the binding capacity of the original AAV9 to said specified chromatography material. A variant of an adeno-associated virus may for example be obtained by spontaneous mutation, or by engineered modification (i.e., obtained by human interaction), of one or more nucleotides of the genome of the adeno-associated virus. This definition applies to all adeno-associated virus serotypes, such as adeno-associated virus serotype 9 (AAV9), adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), and adeno-associated virus serotype 10 (AAV10).

In the present context of separation technology within the field of bioprocessing, a target molecule may be defined by its molecular weight and/or by its hydrodynamic diameter. Non-limiting examples are target molecules having a molecular weight of from about 50 kDa (e.g., a Fab fragment, about 55 kDa), such as target molecules having a molecular weight of about 140 kDa (e.g, a monoclonal antibody, about 150 kDa), such as target molecules having a molecular weight of about 2000 kDa (e.g., adeno-associated virus capsid, about 3900 kDa). Further non-limiting examples are target molecules having a hydrodynamic diameter of from about 7 nm (e.g., a Fab fragment, about 7 nm), such as about 10 nm (e.g, a monoclonal antibody, about 10 nm), such as about 20 nm, such as about 25 nm (e.g., adeno-associated virus capsid, about 20-25 nm).

Correspondingly, the first average pore diameter of the core and the second average pore diameter of the surrounding layer of the presently disclosed chromatography particle may suitably be defined by the molecular weight and/or the hydrodynamic diameter of a target molecule, the diffusion of which the average pore diameter is intended to exclude or permit, respectively.

Accordingly, the first average pore diameter may exclude diffusion of a target molecule having a molecular weight of from about 50 kDa, such as about 140 kDa, such as about 2000 kDa. Alternatively, or additionally, the first average pore diameter may exclude diffusion of a target molecule having a hydrodynamic diameter of from about 7 nm, such as about 10 nm, such as about 20 nm, such as about 25 nm.

In order to exclude diffusion of a target molecule, the pores of the core have an average pore diameter which is substantially smaller than the diameter of the target molecule. In other words, the first average pore diameter is substantially smaller than the diameter of the target molecule. Herein, the term “substantially smaller” is intended to mean at least 10% smaller, such as but not limited to 10%, 15%, 20%, 30%, 40%, 50%, 60%, 70%, 80%, or 90% smaller.

Non-limiting examples include:
(i) the target molecule is an adeno-associated virus capsid, and the first average pore diameter is <15 nm, including but not limited to 13, 12, 11, 10, 7.5, 5, 3, or 1.5 nm;
(ii) the target molecule is a monoclonal antibody, and the first average pore diameter is <10 nm, including but not limited to 9, 8, 7, 6, 5, 4, 3, 2, or 1 nm;
(iii) the target molecule is an antigen-binding fragment of an antibody, and the first average pore diameter is <5 nm, including but not limited to 4, 3, 2, 1, or 0.5 nm;
(iv) the target molecule is a virus-like particle, and the first average pore diameter is <40 nm, including but not limited to 35, 30, 25, 20, 15, 10, or 5 nm;
(v) the target molecule is a lentivirus capsid, and the first average pore diameter is <80 nm, including but not limited to 70, 60, 50, 40, 30, 20, or 10 nm;
(vi) the target molecule is an adenovirus capsid, and the first average pore diameter is <70 nm, including but not limited to 60, 50, 40, 30, 20, or 10 nm; or
(vii) the target molecule is an influenza virus capsid, and the first average pore diameter is <80 nm, including but not limited to 70, 60, 50, 40, 30, 20, or 10 nm.

Further, the second average pore diameter may permit diffusion, at least partly, of a target molecule having a molecular weight of from about 50 kDa, such as about 140 kDa, such as about 2000 kDa. Non-limiting examples of such target molecules are given above. Alternatively, or additionally, the second average pore diameter may permit diffusion, at least partly, of a target molecule having a hydrodynamic diameter of from about 7 nm, such as about 10 nm, such as about 20 nm, such as about 25 nm. Non-limiting examples of such target molecules are given further above.

In order to permit diffusion of a target molecule, the pores of the layer(s) surrounding the core have an average pore diameter which is substantially larger than the diameter of the target molecule. In other words, the second average pore diameter is substantially larger than the diameter of the target molecule. Herein, the term “substantially larger” is intended to mean at least 10% larger, such as but not limited to 10%, 15%, 20%, 30%, 40%, 50%, 60%, 70%, 80%, 90%, or 100% larger, or 2 times, 3 times, 4 times, 5 times, 6 times, 7 times, 8 times, 9 times, or 10 times larger.

Non-limiting examples include:
(i) the target molecule is an adeno-associated virus capsid, and the pores of the layer(s) surrounding the core have an average pore diameter of >25 nm, including but not limited to 30, 50, 75, 100, 150, or 200 nm;
(ii) the target molecule is a monoclonal antibody, and the pores of the layer(s) surrounding the core have an average pore diameter of >11 nm, including but not limited to 15, 30, 50, 75, 100, 150, or 200 nm;
(iii) the target molecule is an antigen-binding fragment of an antibody, and the pores of the layer(s) surrounding the core have an average pore diameter of >7 nm, including but not limited to 10, 15, 30, 50, 75, 100, 150, or 200 nm;
(iv) the target molecule is a virus-like particle, and the pores of the layer(s) surrounding the core have an average pore diameter of >60 nm, including but not limited to 75, 100, 150, or 200 nm;
(v) the target molecule is a lentivirus capsid, and the pores of the layer(s) surrounding the core have an average pore diameter of >100 nm, including but not limited to 120, 150, or 200 nm;
(vi) the target molecule is an adenovirus capsid, and the pores of the layer(s) surrounding the core have an average pore diameter of >90 nm, including but not limited to 110, 150, or 200 nm; or
(vii) the target molecule is an influenza virus capsid, and the pores of the layer(s) surrounding the core have an average pore diameter of >100 nm, including but not limited to 120, 150, or 200 nm.

Further, it is to be understood that a target molecule's ability to diffuse through pores of chromatography particles will not only be determined by the average pore diameter but also will be influenced by parameters such as flow rate and residence time in a chromatography column. All potentially relevant parameters have been taken into account in arriving at the above-specified ranges and non-limiting examples of average pore diameters as representing suitable average pore diameters in relation to separation of the types of target molecules as specified above.

The herein disclosed chromatography particle may have a diameter of from about 20 μm to about 500 μm, such as from about 30 μm to about 150 μm, such as from about 40 μm to about 140 μm, such as from about 60 μm to about 120 μm, or such as from about 100 μm to about 450 μm, such as from about 100 μm to about 400 μm, such as from about 120 μm to about 380 μm, such as from about 150 μm to about 350 μm, or about 20, 30, 40, 50, 60, 70, 80, 90, 100, 110, 120, 130, 140, 150, 160, 170, 180, 190, 200, 250, 300, 350, 400, 450, or 500 μm.

The core may have a diameter of from about 60% to about 97% of the diameter of the chromatography particle, such as about 60, 65, 70, 75, 80, 85, 90, 91, 92, 93, 94, 95, 96, or 97% of the diameter of the chromatography particle.

The layer surrounding the core may have a diameter of from about 3% to about 40% of the diameter of the chromatography particle, such as from about 6% to about 10%, or about 3, 4, 5, 6, 7, 8, 9, 10, 11, 12, 13, 14, 15, 20, 25, 30, 35, or 40% of the diameter of the chromatography particle. It is to be understood that the diameter of the layer surrounding the core is intended to mean the distance consisting of the two radii of the layer surrounding the core, wherein the two radii of the layer surrounding the core extend on opposite sides of the core and wherein each radius extends from the inner end of the layer (i.e., adjacent to the outer end of the core) to the outer end of the layer. In other words, the radius of the layer surrounding the core may be from about 1.5% to about 20% of the diameter of the chromatography particle, such as from about 3% to about 5%, or about 1.5, 2, 2.5, 3, 3.5, 4, 4.5, 5, 6, 7, 8, 9, 10, 11, 12, 13, 14, 15, or 20% of the diameter of the chromatography particle.

A non-limiting example of a chromatography particle which is useful within the context of the present disclosure is a particle having a diameter of about 40 μm, which comprises a core having a diameter of from about 31 μm to about 37 μm, such as about 31, 32, 33, 34, 35, 36, or 37 μm, and a layer surrounding the core having a diameter of from about 3 μm to about 9 μm, such as about 3, 4, 5, 6, 7, 8, or 9 μm.

Another non-limiting example of a chromatography particle which is useful within the context of the present disclosure is a particle having a diameter of about 90 μm, which comprises a core having a diameter of from about 81 μm to about 87 μm, such as about 81, 82, 83, 84, 85, 86, or 87 μm, and a layer surrounding the core having a diameter of from about 3 μm to about 9 μm, such as about 3, 4, 5, 6, 7, 8, or 9 μm.

Yet another non-limiting example of a chromatography particle which is useful within the context of the present disclosure is a particle having a diameter of about 300 μm, which comprises a core having a diameter of from about 291 μm to about 297 μm, such as about 291, 292, 293, 294, 295, 296, or 297 μm, and a layer surrounding the core having a diameter of from about 3 μm to about 9 μm, such as about 3, 4, 5, 6, 7, 8, or 9 μm.

As schematically illustrated by FIG. 2, the above-described layer (30) surrounding the core (20) may be a first layer and the chromatography particle (10) may further comprise a second layer (40) surrounding the first layer (30), wherein the second layer (40) has a third average pore diameter which is different from the first average pore diameter and the second average pore diameter. The third average pore diameter may permit a higher degree of diffusion of the target molecule (i.e., through the pores of the second layer) than the degree of diffusion permitted by the second average pore diameter (i.e., through the pores of the first layer).

The chromatography particle may be substantially spherical, elongated or irregularly formed. It is to be understood that FIG. 1 is a schematic illustration of one embodiment, and that FIG. 2 is a schematic illustration of another embodiment, of the chromatography particle which is useful within the context of the present disclosure, and that chromatography particles having other form than substantially spherical may alternatively be used.

The chromatography particle may be made from an organic or inorganic material. In one embodiment, the chromatography particle is prepared from a native polymer, such as cross-linked carbohydrate material, e.g. agarose, agar, cellulose, dextran, chitosan, konjac, carrageenan, gellan, alginate, pectin, starch, etc. The native polymer chromatography particles are easily prepared and optionally cross-linked according to standard methods, such as inverse suspension gelation (S. Hjerten). In an especially advantageous embodiment, the chromatography particle is a kind of relatively rigid but porous agarose, which is prepared by a method that enhances its flow properties, see e.g. U.S. Pat. No. 6,602,990. In an alternative embodiment, the chromatography particle is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g. “Styrene based polymer supports developed by suspension polymerization” (R. Arshady). Native or synthetic porous polymer chromatography particles are also available from commercial sources, such as Cytiva, Sweden. In yet an alternative embodiment, the chromatography particleis prepared from an inorganic polymer, such as silica. Inorganic porous chromatography particles are well known in this field and easily prepared according to standard methods. The chromatography particle may be of natural or synthetic origin, preferably a polysaccharide, such as agarose. The chromatography particles may be dried (for example, dried agarose particles), which upon use are soaked in liquid to retain their original form.

The term “separation matrix” is used herein to denote a material comprising a support to which one or more ligands comprising functional groups have been coupled. The functional groups of the ligand(s) bind compounds, herein also called analytes or target molecules, which are to be separated from a liquid sample and/or which are to be separated from other compounds present in the liquid sample. In the context of the present disclosure, the support of the separation matrix comprises or consists of the presently disclosed chromatography particles as described in detail herein. A separation matrix may further comprise a compound which couples the ligand(s) to the support. The terms “linker”, “extender”, and “surface extender” may be used to describe such a compound, as further described below. The term “resin” is sometimes used for a separation matrix in this field. The terms “chromatography material” and “chromatography matrix” are used herein to denote a type of separation matrix.

The separation matrix may be contained in any type of separation device, as further defined elsewhere herein. As a non-limiting example, a chromatography material may be packed in a chromatography column, before adding a liquid sample to the chromatography material being contained in the chromatography column.

Accordingly, also useful for the purposes of the present disclosure is a chromatography column comprising a plurality of the presently disclosed chromatography particles, or the presently disclosed separation matrix, as described in detail elsewhere herein.

Optionally the chromatography column may be a bioprocessing chromatography column, including but not limited to a fast protein liquid chromatography (FPLC) column.

Optionally, the chromatography column may be a disposable chromatography column.

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”. Herein, the molecules in a liquid sample which are of interest to separate from the liquid sample, and which interact with a ligand may be referred to as “analyte of interest”, “analyte”, “target molecule”, or “target”.

It is to be understood that the term “liquid sample” as used herein encompasses any type of sample obtainable from a cell culture, or from a fluid originating from a cell culture which fluid is at least partly purified, by any means of separation and purification. For example, the liquid sample may comprise a supernatant obtained from cell fermentation, or the liquid sample may comprise a crude feed.

The term “surface” herein means all external surfaces and includes in the case of a porous support, such as a porous particle, outer surfaces as well as pore surfaces.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a chromatography column after having loaded the liquid sample onto the chromatography column.

The separation matrix may advantageously be a polishing chromatography material, meaning that the chromatography material is applied in a polishing step.

The term “polishing step” refers in the context of liquid chromatography to a final purification step, wherein trace impurities are removed to leave an active, safe product. Impurities removed during the polishing step are often conformers of the target molecule, i.e., forms of the target molecule having particular molecular conformations, or suspected leakage products. A polishing step may alternatively be called “secondary purification step”.

As mentioned above, the separation matrix comprises a support in the form of a plurality of chromatography particles (10), to which one or more ligands have been coupled. Accordingly, the chromatography particle (10) may be said to comprise a ligand for binding to the target molecule. At least the layer (30) surrounding the core (20) of the chromatography particle comprises the ligand. Optionally, the core (20) of the chromatography particle (10) may also comprise the ligand. Whether the core comprises the ligand depends on which method has been used for preparing the chromatography particle, as described further below.

It is to be understood that “a ligand” and “the ligand” is intended to mean a type of ligand and that the singular form of the term may encompass a large number of individual ligands, or specimens, of the same type.

The ligand may be a cationic ligand, an anionic ligand, a hydrophobic interaction ligand, a multimodal ligand, or combinations thereof. It is to be understood that the type(s) of ligand to be used depends on the type(s) of target molecule to be separated from a liquid sample. Different types of target molecules have different binding characteristics, including but not limited to different surface charge, and hence different types of ligands are required to separate different types of target molecules. For example, different serotypes of adeno-associated virus may require different types of ligands in order to achieve a satisfactory separation of fully packaged capsids from not fully packaged capsids.

Below, non-limiting examples of previously known chromatography materials comprising non-limiting examples of such different types of ligands are described. Any one of these different types of ligands, or combinations thereof, may be coupled to the presently disclosed chromatography particle instead of to the type of support material of the known products.

Capto Q, provided by Cytiva, Sweden (www.cytivalifesciences.com) is a non-limiting example of a strong anion exchange chromatography material having about 100% quaternized amine groups. Capto DEAE (Cytiva, Sweden) is a non-limiting example of a strong anion exchange chromatography material having a degree of quaternization of the amine groups of about 15%.

Herein, the term “strong anion exchange chromatography material” is intended to mean a chromatography material which comprises a ligand comprising a quaternized amine group. A quaternary amine group is a strong anion exchange group, which is always positively charged irrespective of to which pH it is subjected. For DEAE-based types of chromatography materials, the degree of quaternization of the amine group may vary among the amine groups included in a chromatography material. A degree of quaternization of the amine group of from about 12% to about 100% globally in a chromatography material is generally considered to result in a chromatography material which behaves like a strong anion exchange chromatography material since these at least 12% of all amine groups are always charged. In contrast to quaternized amine groups, almost all other ionic exchange groups are weak, i.e., their charge varies from fully charged to not charged within a reasonable range of pH used (such as pH 2-11) and having a neutral charge (same amount of + and − charges) at pl.

A chromatography material comprising a carboxymethyl group (abbrev. CM) as the ligand is a non-limiting example of a weak cation exchange chromatography material, provided by Cytiva, Sweden.

A non-limiting example of a strong cation exchange material is Capto S ImpAct (Cytiva, Sweden), which comprises a sulfonate (S) as the strong cation exchanger ligand.

Another non-limiting example of a strong cation exchange material is Capto SP ImpRes (Cytiva, Sweden), which ligand comprises a sulfopropyl (SP) group.

A non-limiting example of a multimodal anion exchange chromatography material comprising hydrophobic groups is a chromatography resin called Capto adhere, or Capto adhere ImpRes (Cytiva, Sweden). The multimodal anion exchange ligand is a N-benzyl-N-methyl ethanol amine ligand coupled to a support, wherein said support is linked to the nitrogen atom of the ligand through a linker. This ligand and chromatography materials comprising this ligand are further described for example in WO2006043896 A1, hereby incorporated by reference in its entirety.

Capto MMC, or Capto MMC Impres is a non-limiting example of a multimodal weak cation exchange chromatography material (Cytiva, Sweden). The ligand contains a carboxylic group. Thus, its features partly resemble a weak cation exchanger. Hydrogen bonding and hydrophobic interactions are also involved.

Further, a non-limiting example of a hydrophobic interaction chromatography material is the aromatic phenyl Sepharose 6 Fast Flow (Cytiva, Sweden) and another non-limiting example of a hydrophobic interaction chromatography material is the aliphatic butyl Sepharose 4 Fast Flow (Cytiva, Sweden).

Irrespective of which type of ligand (i.e., cationic ligand, an anionic ligand, a hydrophobic interaction ligand, a multimodal ligand, or combinations thereof) is chosen, the ligand may optionally be connected to the surface of the presently disclosed chromatography particle via a linker, i.e., the coupling of the ligand to the particle may be provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person.

The ligand may optionally be coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. The function of linkers, especially extenders, is to increase the surface area of the support and to enable functionalization thereof. For a more detailed description of possible chemical structures, see for example U.S. Pat. No. 6,428,707, which is hereby incorporated by reference in its entirety. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl- and polymethacrylamides, polyvinyl ethers etc. Typical biopolymers are polysaccharides, such as starch, cellulose, dextran, agarose.

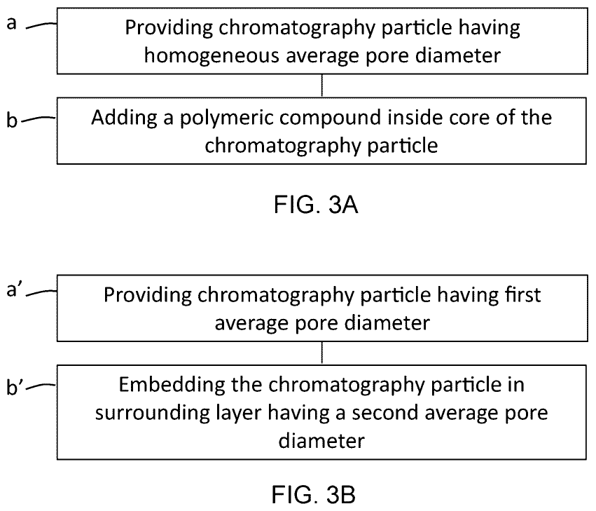

According to a second aspect, the present disclosure provides a method for preparing a separation matrix according to the first aspect, the method comprising preparing a plurality of chromatography particles (10), wherein the method comprises either steps (a) and (b) as illustrated by the flow chart of FIG. 3a, or steps (a′) and (b′) as illustrated by the flow chart of FIG. 3b:
(a) providing a chromatography particle having a homogeneous average pore diameter as starting material, wherein the homogeneous average pore diameter at least partly permits a target molecule to diffuse through the pores of the chromatography particle, throughout the entire structure or volume of the chromatography particle, and
(b) adding a polymeric compound inside a core (i.e., the inner part or central part) of the chromatography particle provided in step (a), thereby decreasing the average pore diameter of the core compared to the homogeneous average pore diameter of the chromatography particle provided in (a),
thereby obtaining a chromatography particle comprising a core and a layer surrounding the core, wherein the core has a first average pore diameter and the layer surrounding the core has a second average pore diameter, wherein the second average pore diameter corresponds to the homogeneous average core diameter of the chromatography particle provided in (a);
   or:
(a′) providing a chromatography particle having a first average pore diameter, which excludes diffusion of a target molecule through the pores of the chromatography particle, throughout the entire structure or volume of the chromatography particle, and
(b′) embedding the chromatography particle provided in step (a′) in a surrounding layer having a second average pore diameter;
   wherein the first average pore diameter and the second average pore diameter are different from each other, wherein the second average pore diameter is at least 1.5 times higher than the first average pore diameter, wherein the first average pore diameter excludes diffusion of a target molecule through the pores of the core and wherein the second average pore diameter at least partly permits diffusion of the target molecule through the pores of the layer surrounding the core.

Herein, the term “homogeneous average pore diameter” is intended to mean that a particle having a homogeneous average pore diameter has a homogeneous (or essentially the same) average pore diameter throughout its entire structure or volume. A chromatography particle having a homogeneous average pore diameter, which is to be provided as starting material in step (a) of the method according to the second aspect, shall have an average pore diameter which permits a target molecule to diffuse, completely or at least partly, through the pores of the particle, throughout the entire structure or volume of the particle.

Further, step (b) of the above-described method according to the second aspect may comprise either (i) or (ii):
(i) coupling of a polymeric molecule to the core of the particle; or
(ii) polymeric grafting, comprising adding monomers to the core of the particle, which monomers are allowed to polymerize inside the core, thereby creating a polymeric molecule inside the core;
   thereby decreasing the average pore diameter of the core.

To decrease the average pore diameter of the core of a particle having a homogeneous average pore diameter, a polymeric molecule may be coupled inside the core of that particle. For example, the average pore diameter may be decreased by a factor of 2 or more, such as by a factor of 3, 4, 5, 6, 7, 8, 9, or 10. For example, a polymeric natural molecule, such as dextran (e.g., Dextran T5 (molecular weight 5 kDa), or Dextran T3.5 (molecular weight 3.5 kDa)), or a polymeric synthetic molecule, for example polyvinyl alcohol or polyvinylpyrrolidone or a combination thereof, can be coupled to the core to decrease the average pore diameter. Alternatively, a polymeric molecule can be generated directly in the core of the particle via polymeric grafting, i.e., by adding monomers to the core, which monomers are then allowed to polymerize inside the core of the particle.

Accordingly, the core of the chromatography particle may comprise a polymeric compound, which decreases the average pore diameter of the core compared to the average pore diameter of the layer(s) surrounding the core. Optionally, such a polymeric compound may be dextran or a synthetic polymer.

The chromatography material used in Example 1 herein comprises a support in the form of substantially spherical chromatography particles or beads, more precisely particles called ImpRes (Cytiva, Sweden), which have a diameter of about 40 μm, as mentioned further above, and which for the purpose of the present disclosure have been modified to obtain a lower average pore diameter in the core than in the layer surrounding the core of the particles. The unmodified ImpRes particle is a non-limiting example of a particle having a homogeneous average pore diameter which at least partly permits diffusion of target molecules of approximately the same size as adeno-associated virus capsids throughout its entire structure or volume. As mentioned above, dextran having a molecular weight of 140-225 kDa, and a hydrodynamic diameter of 20-25 nm (i.e., a diameter of the same size as adeno-associated virus capsids), can be used to evaluate the degree of diffusion of adeno-associated virus capsids within the pores of the particles. The unmodified ImpRes particles have a Kd for dextran of between 0.3 and 0.61, indicating that the capsids will have the possibility to diffuse in approx. 30-60% of the pores.

An ImpRes particle may be modified by coupling or grafting a polymeric molecule inside its core, to create a particle having a lower average pore diameter in its core than in its layer(s) surrounding the core, thereby obtaining a chromatography particle which is useful for the purposes of the present disclosure.

The chromatography material used in Example 2 herein comprises a support in the form of substantially spherical chromatography particles or beads, more precisely particles called Sepharose FF (Cytiva, Sweden), which have a diameter of about 90-100 μm, and which for the purpose of the present disclosure have been modified to obtain a lower average pore diameter in the core than in the layer(s) surrounding the core of the particles. The unmodified Sepharose FF particle is a non-limiting example of a particle having a homogeneous average pore diameter which at least partly permits diffusion of target molecules of approximately the same size as monoclonal antibodies throughout its entire structure or volume. The unmodified Sepharose FF particles have a Kd for dextran 50 k (i.e., dextran having a molecular weight of about 50 000 Da and a diameter of approx. the same size as an IgG antibody) of between 0.5 and 0.6, indicating that monoclonal antibodies will have the possibility to diffuse in approx. 50-60% of the pores.

A Sepharose FF particle may be modified by coupling or grafting a polymeric molecule inside its core, to create a particle having a lower average pore diameter in its core than in its layer(s) surrounding the core, thereby obtaining a chromatography particle which is useful for the purposes of the present disclosure.

A chromatography particle comprising a core and a layer surrounding the core, wherein the layer surrounding the core at least partly permits diffusion of a target molecule and the core excludes diffusion of the same type of target molecule, may herein alternatively be referred to as a “semi-solid bead”, “semi-solid particle”, “semi-solid resin”, “semi-solid matrix”, or similar. A semi-solid particle for use in separating target molecules may for example be produced by preparing a semi-solid particle according to any one of the methods according to the second aspect as described above, and by coupling to the particle of a ligand having affinity for the target molecules. Coupling of the ligand having affinity for the target molecule may be performed before or after preparing the semi-solid particle according to the method according to the second aspect.

Since the core of the particle is occupied by polymeric molecules there will only be ligands accessible for the target molecules in the layer(s) surrounding the core. Further, since the core of the particle has an average pore diameter which does not permit target molecules diffusing into the core, there will only be diffusion of target molecules in the layer(s) surrounding the core (i.e., close to the surface of the particle). This results in shorter diffusion distances than when using an equally sized particle which has a homogeneous average pore diameter throughout its entire structure or volume. As explained in more detail further above, this means that use of semi-solid particles according to the present disclosure may provide advantageous effects like improved resolution and faster separation compared to equally sized particles having a homogeneous average pore diameter. Since the total surface area of the chromatography material will be decreased when filling the core of the particles with polymeric molecules, the binding capacity of the chromatography material will be reduced. However, in some chromatographic applications, resolution between chromatographic peaks is more important than the binding capacity for target molecules.

Results presented in Example 1 below show that the use of semi-solid beads, when the chromatography material comprises a weak cation exchange chromatography material, improved the separation of fully packaged adeno-associated virus capsids from not fully packaged adeno-associated virus capsids. More particularly, the results show that the resolution of peaks in the chromatogram improved when using semi-solid beads compared to use of beads having homogeneous average pore diameter throughout their entire structure or volume. Thus, it has been demonstrated that the use of semi-solid beads improved the separation between full capsids on the one hand and empty and partially filled capsids on the other hand.

Where the target molecules are monoclonal antibodies (mAbs), as in present Example 2, the layer surrounding the core of the particles shall permit diffusion, at least partly, of monoclonal antibodies. The size of a monoclonal antibody is approx. 150 kD and it is approx. 10-15 nm in diameter. Results presented in Example 2 below show that by use of semi-solid beads, decreased peak width could be obtained when separating different proteins, including monoclonal antibodies, using a linear elution gradient. The results also showed that when increasing flow rate, the peak width and resolution of resolved peaks was not affected as for commercially available chromatography beads SP Sepharose FF (Cytiva, Sweden), which have a homogeneous average pore diameter equal to the average pore diameter of the layer surrounding the core of the semi-solid beads tested in Example 2. The disadvantage using the semi-solid beads was that the dynamic protein binding capacity was decreased. However, as mentioned above, resolution between chromatographic peaks may be more important than the binding capacity for target molecules.

For the purposes of the present disclosure, a method for manufacturing a chromatography column is also contemplated. The method comprises preparing a separation matrix (according to the first aspect of the present disclosure), the method comprising preparing a plurality of chromatography particles, optionally by performing the method according to the second aspect (as described above), followed by adding the separation matrix comprising the plurality of thus prepared chromatography particles, to a chromatography column; and sterilizing the chromatography column comprising the chromatography particles or the separation matrix.

A third aspect of the present disclosure is directed to use of a separation matrix (according to the first aspect, as described in detail elsewhere herein) for preparative applications, such as for separating a target molecule from a cell culture harvest or impurities.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture may comprise intact cells, disrupted cells, a cell homogenate, and/or a cell lysate.

The term “cell culture harvest” is used herein to denote a cell culture which has been harvested and removed from the vessel or equipment, in which the cells have been cultivated.

As described in more detail elsewhere herein, the target molecule, which is to be separated from a cell culture harvest or impurities by use of the presently disclosed chromatography particle, may for example be an adeno-associated virus capsid, a monoclonal antibody, an antigen-binding fragment of an antibody, a virus-like particle, a lentivirus capsid, an adenovirus capsid, or an influenza virus capsid.

A fourth aspect of the present disclosure relates to a method of preparative chromatography for separating one or more target molecules from one or more other compounds in a liquid sample, which method comprises contacting a liquid sample comprising the target molecule(s) and compound(s) with a separation matrix (according to the first aspect) or with a chromatography column comprising such a separation matrix, and which method comprises exposing the liquid sample to a differential pressure (or “delta pressure”) of <5 bar, such as <3 bar, or such as 4.5, 4, 3.5, 3, 2.5, or 2 bar. The term “preparative chromatography” is used here in conjunction with the main application areas of the presently disclosed chromatography particles, as opposed to analytical applications and high-pressure liquid chromatography (HPLC), for which the presently disclosed chromatography particles would not be suitable. Preparative chromatography may herein be defined by the application of a delta column pressure not exceeding 5 bar, preferably not exceeding 3 bar.

Where the method according to the fourth aspect comprises contacting the liquid sample comprising the target molecule(s) with a chromatography column, in which a separation matrix is provided, a mobile phase containing the liquid sample may be passed across said chromatography column by gravity and/or by pumping, and either
(a) the target molecules may be recovered in the flow-through of the chromatography column, in which case the compounds adsorbed to the separation matrix are host cell proteins and/or other impurities, or
(b) the target molecules may be recovered in eluate fractions from the chromatography column, in which case host cell proteins and other impurities are found in the flow-through of the column.

The method according to the fourth aspect may further comprise a step of clarification (e.g., by filtration, centrifugation, or precipitation), optionally also concentration and/or stabilisation, and/or chromatography preceding the above-described step of contacting the mobile phase comprising the target molecule(s) and compound(s) with the separation matrix, or with the chromatography column. Said step of clarification and/or chromatography comprises pre-purifying the target molecule(s), for example by separating them from a cell culture harvest, thereby obtaining a pre-purified liquid sample comprising the target molecule(s). Said pre-purified liquid sample may subsequently be contacted with the separation matrix, or with the chromatography column.

Such a pre-purifying step may alternatively be called a “capture step” and refers in the context of liquid chromatography to the initial step(s) of a separation procedure. Such a pre-purifying step may comprise subjecting the target molecule-containing cell culture harvest to one or more of the following non-limiting examples of purification methods:
(i) affinity chromatography,
(ii) ion exchange chromatography,
(iii) precipitation or tangential flow filtration (TFF), followed by size-exclusion chromatography, such as by use of for example Capto Core 400 chromatography material (Cytiva, Sweden), which combines flow-through of the target molecules with binding of impurities to the chromatography material,
(iv) TFF followed by ion exchange chromatography, and
(v) TFF followed by ion exchange chromatography and Capto Core.

Non-limiting examples of chromatography materials suitable to apply in a pre-purifying step of the method described above include affinity chromatography material, ion exchange chromatography material, and size-exclusion chromatography material, respectively. The chromatography material may be functionalized with a positively charged group, such as a quaternary amino, quaternary ammonium, or amine group, or a negatively charged group, such as a sulfonate or carboxylate group. The chromatography material may be functionalized with an ion exchanger group, an affinity peptide/protein-based ligand, a hydrophobic interaction ligand, an IMAC ligand, or a DNA based ligand such as Oligo dT.

The term “separation device” has its conventional meaning in the field of bioprocessing and is to be understood as encompassing any type of separation device which is capable of and suitable for separating and purifying compounds from a fluid containing by-products from the production of the compounds. A separation device may comprise a separation matrix, as further defined elsewhere herein.

Non-limiting examples of separation devices suitable for use in a capture step, or pre-purification step, as described herein, are filtration apparatuses, chromatography columns and membrane devices. Chromatography columns suitable for use in the capture step may for example be packed with affinity chromatography material, ion exchange chromatography material, mixed mode chromatography material or hydrophobic interaction chromatography material.

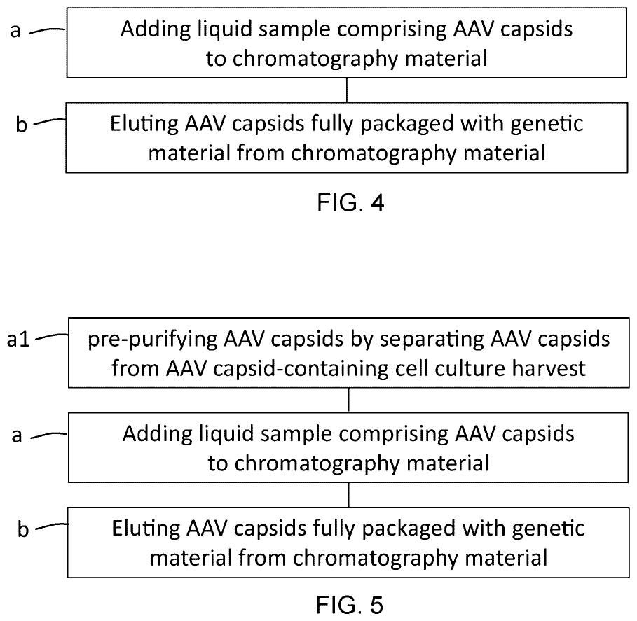

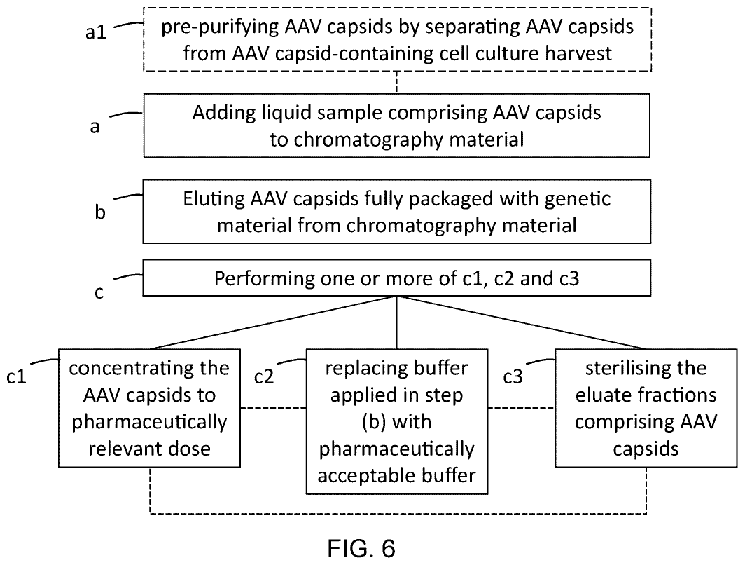

A fifth aspect of the present disclosure is directed to a particular method of preparative chromatography for separating one or more target molecules from one or more other compounds in a liquid sample. More specifically, the present disclosure solves or at least mitigates the problems associated with existing methods for separating fully packaged adeno-associated virus capsids from capsids not fully packaged adeno-associated virus capsids by providing, as illustrated in FIG. 4, a method for separating adeno-associated virus capsids fully packaged with genetic material from adeno-associated virus capsids not fully packaged with genetic material, the method comprising the following steps:
a) adding a liquid sample comprising adeno-associated virus capsids to a weak cation exchange chromatography material,
   wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
   wherein the weak cation exchange chromatography material comprises:
      i. a ligand for binding to the adeno-associated virus capsids; or
      ii. a support, which comprises a separation matrix according to the first aspect of the present disclosure, as described above;
b) eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

Significant advantages of the method according to the fifth aspect of the present disclosure include that it is suitable for large-scale separation of fully packaged capsids from not fully packaged capsids and that it provides an improved ratio of fully packaged to not fully packaged capsids compared to prior art methods. By performing the method, it is possible to obtain a composition which has a ratio of fully packaged capsids to not fully packaged capsids of at least 3:2. More particularly, the presently disclosed method provides an improved ratio of fully packaged capsids to empty capsids, as well as an improved ratio of fully packaged capsids to partially packaged capsids.

Definitions of the following terms may be found further above in this text: “genetic material of interest”, “capsid fully packaged with genetic material”, “capsid not fully packaged with genetic material”, “partially filled capsid”, and “empty capsid”.

Before putting a population of virus particles to use in its intended application, e.g., a therapeutic application, it is desirable (sometimes even required, e.g., due to clinical regulations) to enrich the full capsids, i.e., to increase the percentage of full capsids at the expense of the percentage of partially filled capsids and empty capsids. The percentage of full capsids and empty capsids in a population of capsids can be estimated or analyzed with several methods known in the art. Some of these methods have been briefly described further above.

As mentioned above, in the method for separating fully packaged capsids from not fully packaged capsids, the liquid sample which is added to a chromatography material in step (a) comprises adeno-associated virus capsids of a purity of at least 90%, such as 90%, 91%, 92%, 93%, 94%, 95%, 96%, 97%, 98%, or 99% and of a concentration of at least 10¹², such as 10¹³, 10¹⁴, or 10¹⁵, adeno-associated virus capsids/ml, of which at least 10%, such as 15%, 20%, 25%, 30%, 40%, 50%, 60%, 70%, or 80%, of the adeno-associated virus capsids are adeno-associated virus capsids fully packaged with genetic material. With regard to the purity of adeno-associated capsids in the liquid sample, a purity of at least 90%, such as up to 99%, is intended to mean that at least 90%, such as up to 99%, of the biological material in the liquid sample is represented by adeno-associated capsids (including full, empty, and partially filled capsids) while the remaining up to 10%, such as 1%, is represented by host cell protein and DNA.

The aim of step (b) of the above-disclosed method is to obtain fully packaged capsids of a purity which is as high as possible. A person skilled in the art readily understands that this may be achieved by applying various different separation conditions. Non-limiting examples of separation conditions to obtain fully packaged capsids of a purity as high as possible include separation conditions which allow binding of not fully packaged capsids to the chromatography material, while:
(i) allowing fully packaged capsids to substantially flow through the chromatography material (i.e., fully packaged capsids substantially not binding to the chromatography material), or
(ii) allowing fully packaged capsids to bind to the chromatography material followed by eluting them from the chromatography material. It is to be understood that in the bind-elute process described in item (ii), the fully packaged capsids may be eluted from the chromatography material before or after not fully packaged capsids, depending on which separation conditions are applied.

As mentioned above, there are small differences between fully packaged capsids and not fully packaged capsids in relation to several parameters relevant for purification, e.g., their isoelectric point. This often leads to (at least partial) co-elution of fully packaged and not fully packaged capsids. Accordingly, realistically, the adeno-associated virus capsids eluted in step (b) of the above-disclosed method will not be completely separated into full, empty, and partially filled capsids. However, there will be eluate fractions which comprise a substantially higher percentage of full capsids than in the liquid sample added to the chromatography material in step (a). More particularly, as disclosed above, the adeno-associated virus capsids eluted in step (b), i.e., adeno-associated virus capsids fully packaged with genetic material, are eluted into eluate fractions, which eluate fractions combined comprise at least 50%, such as 55%, 60%, 65%, 70%, 75%, 80%, 85%, or 90%, of the adeno-associated virus capsids of the liquid sample added in step (a), of which at least 60%, such as 65%, 70%, 75%, 80%, 85%, or 90%, of the adeno-associated virus capsids are fully packaged with genetic material. Non-limiting examples of recovery and purification of full capsids achieved by the presently disclosed method are a recovery of at least 50% of the capsids of the liquid sample added in step (a), of which at least 60% are full capsids, such as a recovery of at least 70% of the capsids of the liquid sample added in step (a), of which at least 80% are full capsids. In Example 1 described further below, the results show a recovery of 60-70% of capsids from harvest, of which at least 60% are full capsids, and further shows an improved resolution of partially filled capsids by use of chromatography particles according to the present disclosure, compared to use of chromatography particles having homogeneous average pore diameter throughout their entire volume. At present, there is no large-scale method publicly available which gives as high recovery and purification as the herein disclosed methods.

As mentioned above, the chromatography material applied in the presently disclosed method comprises a weak cation exchange chromatography material, comprising a ligand for binding to the adeno-associated virus capsids and a support, which comprises a separation matrix according to the first aspect of the present disclosure.

The ligand of the weak cation exchange chromatography material may be defined by the following Formula I:

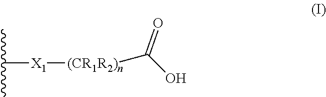

wherein
X₁ is selected from O, S, and NHCO;
n is an integer of from 1 to 4, i.e., 1, 2, 3, or 4; and
each R₁ and R₂ is independently selected from hydrogen, C₁-C₃ alkyl, and OH.

As a non-limiting example, X₁ is O, n is 1, and each R₁ and R₂ is hydrogen.

There are currently available chromatography materials comprising a ligand defined by Formula I, wherein X₁ is O, n is 1, and each R₁ and R₂ is hydrogen, i.e., wherein the ligand is a carboxymethyl group (abbrev. CM), provided by Cytiva, Sweden (www.cytivalifesciences.com).

According to another non-limiting example, X₁ is S, n is 2, and each R₁ and R₂ is hydrogen.

According to yet another non-limiting example, X₁ is NHCO, n is 2 or 3, and each R₁ and R₂ is hydrogen.

According to yet another non-limiting example, X₁ is O, n is 1, R₁ is CH₃, and R₂ is hydrogen.

The density of ligand may be from about 30 to about 110 μmol, such as from about 40 to about 90 μmol, such as from about 50 to about 80 μmol, or about 30, 35, 40, 41, 42, 43, 44, 45, 46, 47, 48, 49, 50, 51, 52, 53, 54, 55, 56, 57, 58, 59, 60, 61, 62, 63, 64, 65, 70, 75, 80, 85, 90, 95, 100, 105 or 110 μmol, of ligand per ml of the weak cation exchange chromatography material.

Steps (a) and (b) of the above-disclosed method according to the fifth aspect of the present disclosure may comprise applying a buffer having a pH of from about 2.0 to about 12.0, such as from about 3.0 to about 9.0, such as from about 4.0 to about 6.0, such as about 4.5, or about 2.0, 2.5, 3.0, 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10.0, 10.5, 11.0, 11.5, or 12.0.

Said buffer is suitably selected from buffers generally recommended for cation exchange chromatography, and may for example comprise citric acid, lactic acid, acetic acid, phosphate, MES (2-(N-Morpholino)ethanesulfonic acid), ADA (N-(2-Acetamido)-2-iminodiacetic acid), MOPS (3-(N-Morpholino)propanesulfonic acid), HEPES (N-(2-Hydroxyethyl)piperazine-N′-(2-ethanesulfonic acid), tris(hydroxymethyl)amino-methane (i.e., Tris), or 1,3-bis(tris(hydroxymethyl)methylamino) propane (i.e., bis-Tris propane).

A person skilled in the art is able to choose a suitable concentration for any one of the above-listed buffers. Non-limiting examples of buffers applied in steps (a) and (b) of the method according to the fifth aspect, where the chromatography material is a weak cation exchange chromatography material, may comprise from about 10 to about 100 mM of acetate, such as from about 20 to about 60 mM of acetate, or about 10, 15, 20, 25, 30, 35, 40, 45, 50, 55, 60, 65, 70, 75, 80, 85, 90, 95, or 100 mM, of acetate. Such buffers may for example have a pH of from about 2.0 to about 12.0, such as from about 3.0 to about 9.0, such as from about 4.0 to about 6.0, such as about 4.5, or about 2.0, 2.5, 3.0, 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10.0, 10.5, 11.0, 11.5, or 12.0.

Step (b) of the method according to the fifth aspect may comprise applying a buffer, optionally one of the buffers mentioned above, wherein the buffer comprises a compound which improves separation between capsids fully packaged with genetic material and capsids not fully packaged with genetic material. This compound may or may not be present in a buffer applied in step (a) of the method according to the fifth aspect. Without being bound by theory, such a compound may for example improve separation by influencing interactions between capsid and ligand or interactions between capsid and capsid. Said compound which improves separation may for example be selected from a carbohydrate, a divalent metal ion, and a detergent.

Where said compound which improves separation is a carbohydrate, it may for example be selected from sucrose, sorbitol, and a polysaccharide.

Where said compound which improves separation is a divalent metal ion, it may for example be selected from Mg²⁺, Fe²⁺, and Mn²⁺. The metal ion may be present in the form of a salt, optionally in combination with for example chloride ions or sulphate ions. A non-limiting example of a suitable metal salt to include in the buffer of step (b) is MgCl₂. Non-limiting examples of suitable concentrations of MgCl₂ include from about 0.5 to about 30 mM of MgCl₂, such as from about 1 to about 20 mM, such as from about 2 to about 10 mM, or about 0.5, 1.0, 1.5, 2.0, 2.5, 3, 4, 5, 6, 7, 8, 9, 10, 11, 12, 13, 14, 15, 16, 17, 18, 19, 20, 25, or 30 mM, of MgCl₂.

Where said compound which improves separation is a detergent, it may for example be selected from poloxamer, such as poloxamer 188 or Pluronic™ F68, and polysorbate, such as Tween 20 or Tween 80.

In the above-described method according to the fifth aspect, step (b) may comprise applying a buffer, optionally one of the buffers mentioned above, wherein the buffer comprises a compound which may help eluting capsids bound to the chromatography material. This compound is not present in a buffer applied in step (a) of the method according to the fifth aspect. Non-limiting examples of such a compound is a salt, such as a salt of a monovalent metal ion, e.g., NaCl, LiC, KCl, or other equivalent metal salt suitable to use for salt elution, as is well known in the art. Non-limiting examples of suitable concentrations of NaCl include from about 50 mM to about 2M of NaCl, such as about 50, 100, 150, 200, 250, 500, 600, 700, 800, 900, 1000, 1500, or 2000 mM, of NaCl. Further, step (b) may comprise applying a gradient of such a compound to improve elution of the adeno-associated virus capsids fully packaged with genetic material from the chromatography material.

Non-limiting examples of suitable buffers to be applied in step (b), where the chromatography material is a weak cation exchange chromatography material, may comprise (i) 25 mM acetate, 10 mM MgCl₂, 500 mM NaCl, pH 4.5, (ii) 25 mM acetate, 2 mM MgCl₂, 800 mM NaCl, pH 4.5, (iii) 25 mM acetate, 500 mM NaCl, pH 4.5, (iv) 25 mM acetate, 500 mM NaCl, pH 4.5, or (v) 25 mM acetate, 500 mM NaCl, 10 mM MgCl₂, pH 4.5, (vi) 50 mM acetate, 500 mM NaCl, pH 4.5.

The liquid sample added in step (a) of the herein disclosed method according to the fifth aspect may advantageously be a pre-purified liquid sample.

The present disclosure further provides a method for separating fully packaged adeno-associated virus capsids from not fully packaged adeno-associated virus capsids, comprising performing the method as shown in FIG. 4 and as described in detail above, the method further comprising a step (al) which comprises pre-purifying adeno-associated virus capsids by separating adeno-associated virus capsids from an adeno-associated virus capsid-containing cell culture harvest, as illustrated in FIG. 5, thereby obtaining a pre-purified liquid sample comprising adeno-associated virus capsids, before adding said pre-purified liquid sample comprising adeno-associated virus capsids to the chromatography material according to step (a) of the method as shown in FIG. 4 and FIG. 5, respectively.

As described further above, such a pre-purifying step (al) may alternatively be called a “capture step”. Most commonly, a capture step includes clarification (e.g. by filtration, centrifugation, or precipitation), and normally also concentration and/or stabilisation of the sample, and a significant purification from soluble impurities, for example by applying chromatography after the clarification, concentration, and stabilisation of sample. After the capture step, an intermediate purification may follow, which further reduces remaining amounts of impurities such as host cell proteins, DNA, viruses, endotoxins, nutrients, components of a cell culture medium, such as antifoam agents and antibiotics, and product-related impurities, such as aggregates, misfolded species, and aggregates.

Such a pre-purifying step may comprise subjecting the adeno-associated virus capsid-containing cell culture harvest to one or more of the following non-limiting examples of purification methods:
(i) affinity chromatography,
(ii) ion exchange chromatography,
(iii) precipitation or tangential flow filtration (TFF), followed by size-exclusion chromatography, such as by use of for example Capto Core 400 chromatography material (Cytiva, Sweden), which combines flow-through of the capsids with binding of impurities to the chromatography material,
(iv) TFF followed by ion exchange chromatography, and
(v) TFF followed by ion exchange chromatography and Capto Core.

As mentioned further above, adeno-associated viruses are approx. 20-25 nm in diameter. Since a capsid is the shell of a virus particle, and since adeno-associated viruses do not have a lipoprotein bilayer envelope surrounding the capsid, the size of an adeno-associated virus capsid is approx. 20-25 nm in diameter.

Accordingly, the layer(s) surrounding the core of the plurality of chromatography particles comprised by the separation matrix applied in the method according to the fifth aspect of the present disclosure may suitably comprise an average pore diameter which is >25 nm, i.e., larger than the diameter of the adeno-associated virus capsids to be separated, thereby enabling diffusion of capsids within the layer(s) surrounding the core. It is to be understood that for the specific purpose of separating adeno-associated virus capsids, an average pore diameter of >25 nm may be of any size >25 nm, including but not limited to 30, 50, 75, 100, 150, or 200 nm.

In contrast, the average pore diameter of the core does not permit diffusion of adeno-associated virus capsids. Based on the size of the adeno-associated viruses, the core may suitably comprise an average pore diameter which is <20 nm, i.e., smaller than the diameter of the adeno-associated virus capsids to be separated, thereby disallowing, or hindering diffusion of capsids within the core.

Preferably, the core may comprise an average pore diameter which is substantially smaller than the size of adeno-associated viruses, such as a diameter of <15 nm, such as 10 nm or 5 nm.

A chromatography particle having a lower average pore diameter in its core than in its layer(s) surrounding the core may, for example, be prepared by performing the method according to the second aspect of the present disclosure.

The herein disclosed method for separating fully packaged adeno-associated virus capsids from not fully packaged adeno-associated virus capsids may further comprise subjecting the eluate fractions comprising adeno-associated virus capsids fully packaged with genetic material, eluted in step (b) of the method as described above, to one or more of the following steps:
c1) concentrating the adeno-associated virus capsids to a pharmaceutically relevant dose,
c2) replacing a buffer applied in step (b) of the method with a pharmaceutically acceptable buffer, and/or
c3) sterilizing the eluate fractions comprising adeno-associated virus capsids, thereby obtaining a pharmaceutical composition comprising adeno-associated virus capsids.

A person skilled in the art understands that the pharmaceutically relevant dose will depend on various factors such as, but not limited to, the disease or disorder to be treated as well as the weight and condition of the subject to be treated with a pharmaceutical composition.

Pharmaceutically acceptable buffers are well known in the art and can easily be chosen by the skilled person.

For the resulting composition to fulfil all regulatory requirements for pharmaceutical compositions, normally all of the above-listed three steps c1-c3 have to be performed.

In the above-disclosed method according to the fifth aspect of the present disclosure, the adeno-associated virus capsids may advantageously be capsids of adeno-associated virus serotype 9 (AAV9) or a variant thereof.

The term “variant” in relation to an adeno-associated virus serotype 9 (AAV9) is intended to mean a modified or engineered AAV9, in which the capsid structure has been modified to improve clinical performance, for example towards a specific target organ. An AAV9 variant comprises capsid parts of AAV9 and may additionally comprise capsid parts of other AAV serotypes than AAV9, such as AAV5. However, an AAV9 variant as referred to herein must retain a significant structural similarity to a non-modified AAV9 capsid, such as retaining at least 50%, such as 60%, 70%, 80%, or 90%, of the external surface structure of a non-modified AAV9 capsid. In the context of purification or separation of a variant of AAV9, a “variant” is herein defined as an adeno-associated virus which has a functionally equivalent binding capacity to the ligand of a specified chromatography material, compared to the binding capacity of the original AAV9 to said specified chromatography material. The specified chromatography material may, for example, be a weak cation exchange chromatography material as disclosed in more details elsewhere herein. A variant of an adeno-associated virus may for example be obtained by spontaneous mutation, or by engineered modification (i.e., obtained by human interaction), of one or more nucleotides of the genome of the adeno-associated virus.

The present disclosure is further useful for performing a method for preventing or treating a disease or disorder related to an organ or tissue in a subject, comprising administering to the subject a pharmaceutical composition comprising adeno-associated virus capsids obtained by performing the above-disclosed separation method comprising one or more of steps c1-c3 (as described in detail above), in which pharmaceutical composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

Said method for preventing or treating a disease or disorder related to an organ or tissue may suitably comprise gene therapy. It is to be understood that the therapeutically relevant gene(s) or genetic material is present in the adeno-associated virus capsids fully packaged with genetic material comprised by the pharmaceutical composition which is to be administered to the subject.

In the above-described method for preventing or treating a disease or disorder related to an organ or tissue, the adeno-associated virus capsids may advantageously be capsids of adeno-associated virus serotype 9 (AAV9) or a variant thereof.

In the above-described method for preventing or treating a disease or disorder related to an organ or tissue, the organ or tissue may optionally be selected from the central nervous system, heart, liver, lung, and skeletal muscle, particularly where the adeno-associated virus capsids are capsids of adeno-associated virus serotype 9 (AAV9) or a variant thereof.

It is to be understood that the above-mentioned tissue types are non-limiting examples of organs and tissue types for which treatment by administration of a pharmaceutical composition comprising adeno-associated virus capsids, in particular capsids of adeno-associated virus serotype 9 (AAV9) or a variant thereof, may be applicable.

A non-limiting example of a disease or disorder related to an organ or tissue is spinal muscular atrophy, for which a method of treatment or prevention may suitably be performed by administration of adeno-associated virus, in particular AAV9.

Another non-limiting example of a disease or disorder related to an organ or tissue is inherited retinal dystrophy, for which treatment or prevention may suitably be performed by administration of adeno-associated virus, in particular AAV2.

A person of skill in the art understands that the pharmaceutical composition must be administered in a pharmaceutically effective amount or dose to the subject to achieve the desired medical effects. Amounts and doses which are pharmaceutically effective depend on various factors such as, but not limited to, the disease or disorder to be treated as well as the weight and condition of the subject to be treated.

The present disclosure further provides, according to a sixth aspect, a composition comprising adeno-associated virus capsids obtained by performing the method for separating fully packaged adeno-associated virus capsids from not fully packaged adeno-associated virus capsids (i.e., the method according to the fifth aspect) as described in detail above, in which composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

The present disclosure is further useful for providing a pharmaceutical composition comprising adeno-associated virus capsids obtained by performing the above-disclosed separation method comprising one or more of steps c1-c3 (as described in detail above), in which pharmaceutical composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

The above-described pharmaceutical composition may be for use in therapy, optionally for use in gene therapy.

In the above-described pharmaceutical composition for use in therapy, such as gene therapy, the adeno-associated virus capsids may advantageously be capsids of adeno-associated virus serotype 9 (AAV9) or a variant thereof.

Further, the pharmaceutical composition may advantageously be for use in the prevention or treatment of a disease or a disorder related to an organ or tissue selected from the central nervous system, heart, liver, lung, and skeletal muscle.

Devices or compositions “comprising” one or more recited components may also include other components not specifically recited. The term “comprising” includes as a subset “consisting essentially of” which means that the device or composition has the components listed without other features or components being present. Likewise, methods “comprising” one or more recited steps may also include other steps not specifically recited.

The singular “a” and “an” shall be construed as including also the plural.

### Example 1: Separation of AAV9 Capsids on Weak Cation Exchange Chromatography Materials Comprising Semi-Solid Particles

### Preparation of Chromatography Materials

### a) Carboxymethyl (CM) Ligand Prototypes (Resin 1, 2 and 3) on Beads Having Homogeneous Average Pore Diameter

### General Procedure:

Highly cross-linked agarose base matrix (40 mL of beads having homogeneous average pore diameter) was washed with water (2 gel volumes (GV)) 5 times, drained and transferred to a 100 ml three-neck RB flask and then 2.5 ml of water was added. The gel mixture was kept in a water bath at room temperature (R.T.). A NaOH solution (50% in water, 7.8 ml) was added and the mixture was kept stirring with a mechanical stirrer. After 30 mins sodium chloroacetate was added in one portion (respectively, 13.2 g for resin 1; 6.6 g for resin 2 and 19.88 g for resin 3). The reaction is exothermic, and the temperature should be well kept at 20° C. After the dissolution of sodium chloroacetate, the water bath temperature was raised up to 33° C. The reaction was kept stirring at 33° C. for 16 hours. Water (10 mL) was then added, and the solution was cooled down to R.T. Acetic acid (60%) was added to adjust the pH of the solution to 6.8-7.2. The resulting gel was washed 3 times with water (1 GV).

Titration of the resulting resins gave an ionic capacity (i.e., ligand density) of 45 μmol/mL for resin 1, 30 μmol/mL for resin 2 and 56 μmol/mL for resin 3.

### b) Carboxymethyl (CM) Prototype on Semi-Solid Beads (i.e., an Example of Chromatography Particles as Comprised by a Separation Matrix According to the First Aspect of the Present Disclosure)

### b.1) Allylation of Cross-Linked Agarose Base Matrix

Highly cross-linked agarose base matrix (300 mL of beads having homogeneous average pore diameter) was washed 10×GV with distilled water. The gel was then sucked dry and transferred to a 2000 mL round bottom flask. 300 ml of 50% NaOH was added, mechanical propeller stirring (250 rpm) was applied and the flask was immersed into a water bath at 50° C. After 30 minutes 93.2 mL of allyl glycidyl ether (AGE) was added. The reaction progressed for 18 h. The gel was washed 1×GV with distilled water, 5×GV with ethanol and then 8×GV with distilled water. Titration gave an allyl level of 266 μmol/mL of resin.

### b.2) Partial Activation (Outer Part)

The allylated resin from above (70.1 mL) was transferred to a 1000 mL round-bottomed flask. 350 mL of distilled water was added and mechanically stirred. A solution of 529 μL bromine in 83.3 mL of water was prepared. The bromine solution was added slowly during approximately 2-3 minutes under stirring. After 20 minutes at room temperature the gel was washed with 10×GV distilled water. Titration of remaining allyl groups indicated a shell (i.e., an outer layer; a layer surrounding the core of the bead) of 3 μm.

### b.3) Coupling of 3-Mercaptopropionic Acid

Partially activated gel of b.2) (30.03 mL) was transferred to a 100 mL round-bottomed flask. 10.48 mL of distilled water was added and mechanical stirring was applied. NaOH (5.42 mL of 50% solution) was added followed by 3-mercaptopropionic acid (120.22 μL). The flask was left at 50° C. for 18 h. The gel was washed 15×GV with distilled water.

b.4) Allyl-Dextran (Allyl-Dx) Coupling to Allyl Core, Shell Comprising 3-Mercaptopropionic Acid-carboxymethyl ligand:

Solubilization of allyl-Dx: 19.96 g of allyl-Dx was added together with 20 mL of water to a 250 mL round flask with stirring (75 rpm) over night (23 h) at room temperature and then at 30° C. for 110 minutes.

### b.4.a) Bromination of Allyl Core

20 g of resin from b.3), 20.05 mL of water and 0.8 g of NaOAc*3H2O were mixed in an 100 mL E-flask.

Bromine water was added until a persistent yellow colour was obtained. The reaction was stirred for minutes before excess bromine was quenched by adding sodium formiate. The gel was then washed 5×GV with water.

### b.4.b) Coupling of Allyl-Dx

The brominated gel of b.4.a) was added to the allyl-Dx solution and the temperature was raised to 50° C. An extra 5 mL of water was added. After 90 minutes, 2.6 mL of 50% NaOH and 106 mg of NaBH4 were added. The reaction progressed for 18 h under stirring. The mixture was then diluted with water and the gel was washed 10×GV with water. The dry weight of the gel was measured.

b.5) Polymerisation of Allyl-Dx Core with Vinylpyrrolidone/ADBA:

Resin from b.4.b) (18.8 mL) of was added with water (122.33 g) to a 250 mL flask. 0.94 g of 2,2′-Azobis(2-amidinopropane) dihydrochloride (AAPH) initiator and 46.18 g of vinylpyrrolidone were added to the flask. After bubbling with nitrogen (0.1 NL/min) for 5 min the flask was left at 55° C. for 19 h. The very viscous solution was then diluted and washed on a glass filter 5×GV with water, at least 5×2GV with EtOH and then 10×5GV with water.

Titration gave an ionic capacity (i.e., ligand density) of 45 μmol/mL of semi-solid resin.

Accordingly, the ligand density of the layer surrounding the core of the semi-solid beads described above under (b) is comparable to the ligand density of resin 1 described above under (a).

### Equipment and Samples

All resins were packed in Tricorn 5 column (2 mL), 10 cm bead height and run using Agilent Bio-Inert 1260 system or an AKTA pure P25 system with a flow rate of 1 mL/min, using linear gradient and different mobile phase systems according to Results below. For all different resins, the sample applied was approx. 1×10¹³ virus capsids of AAV9, pre-purified with affinity chromatography and containing both full and empty capsids (>15% full capsids). Detection was made with fluorescence (excitation at 280 nm, emission 348 nm) or UV (280 and 260 nm).

### Results

For comparative purposes, currently available (i.e., prior art) cation and cation/multimodal resins (SP Sepharose XL, Capto SP ImpRes, Capto SP ImPact and Capto MMC ImpRes, CM Sepharose Fast Flow, and CM Sepharose High performance; all provided by Cytiva, Sweden) were evaluated with 50 mM acetate pH 4.5 and 5.5 with a salt gradient up to 1M NaCl. The results showed no separation and only a single peak when applying AAV9 pre-purified with affinity chromatography (results not shown).

Further, the CM prototype resin 2 as referred to above under (a), having a ligand density of 30 μmol/mL and homogeneous porosity, was run with 25 mM acetate, pH 4.5, 500 mM NaCl, 10 mM MgCl₂ and the results showed a separation of AAV9 full and empty capsids (results not shown).

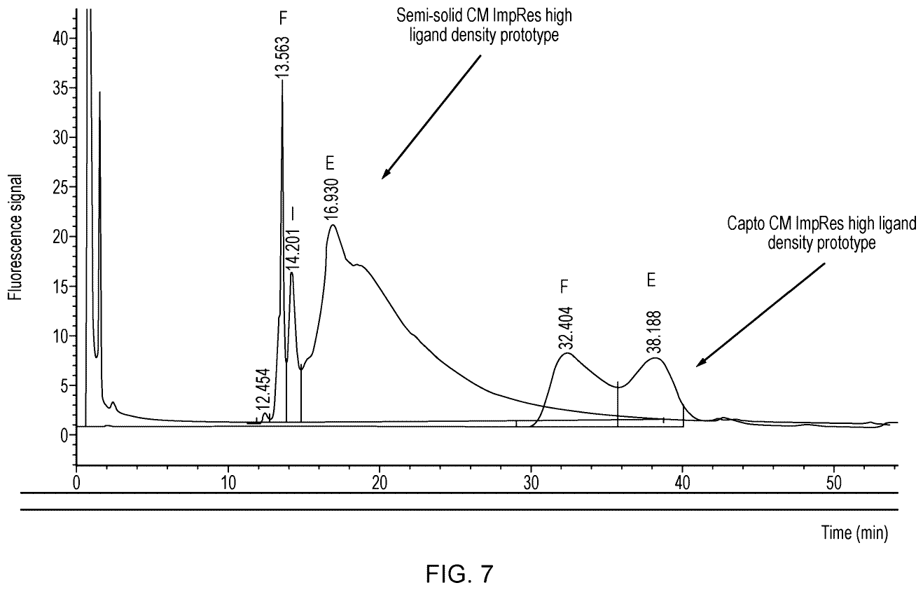

Using CM prototype resin 3 as referred to above under (a), having an increased ligand density of 56 μmol/mL of resin and homogeneous porosity, under the same chromatography conditions as above, the separation performance increased (FIG. 7). The two peaks marked F and E to the right in FIG. 7 show full (F) capsids and empty (E) capsids, respectively, separated on the CM prototype resin 3 having homogeneous porosity. From FIG. 7 it may further be deduced that separation on the CM prototype resin 3 did not result in a clear intermediate peak.

When a semi-solid CM prototype resin with the corresponding higher ligand density (above under (b) referred to as CM prototype on semi-solid bead) was evaluated, even better resolution was achieved (FIG. 7). The three peaks marked F, I, and E to the left in FIG. 7 contain a majority of full (F) capsids, partially filled (I; intermediate) capsids, and empty (E) capsids, respectively, separated on the semi-solid CM prototype resin.

A preparative run with the high ligand density semi-solid CM prototype resin was run on the AKTA system. Pooled peak 1 fractions and pooled peak 3 fractions, respectively, from the run were analyzed using TEM to confirm that peak 1 contains full capsids and peak 3 contains empty capsids (Table 1). The qPCR and ELISA ratio also showed enrichment of full capsids in peak 1 (data not shown).

TABLE 1

| Semi-solid CM | Peak 1 | | Peak 3 | |
|---|---|---|---|---|
| prototype resin | No. of | % of | No. of | % of |
| AAV9 | capsids | capsids | capsids | capsids |
| Full | 46 | 52 | 237 | 25 |
| Intermediate | 14 | 16 | 238 | 25 |
| Empty | 28 | 32 | 478 | 50 |

### Conclusions

In the experiments described above, it was seen that the CM prototype having the highest ligand density (i.e., resin 3) gave better resolution than the CM prototype having lower ligand density (i.e., resin 2). The semi-solid CM prototype (i.e., an example of the chromatography particles comprised by a separation matrix according to the first aspect of the present disclosure) gave an even higher resolution than the CM prototype resin 3. Further, MgCl₂ added in the elution buffer drastically increased resolution when using the semi-solid CM column but not when using any of the CM prototypes (i.e., resin 2 and 3).

### Example 2: Separation of Monoclonal Antibody Variant on Strong Cation Exchange Chromatography Material Comprising Semi-Solid Particles

### Preparation of Chromatography Materials

### a) Sulfonate (S) Prototype on Semi-Solid Beads (i.e., an Example of Chromatography Particles Comprised by a Separation Matrix According to the First Aspect of the Present Disclosure)

### a.1) Allylation of Cross-Linked Agarose Base Matrix

Cross-linked agarose base matrix (300 mL of beads having homogeneous porosity) was washed 10×GV with distilled water. The gel was then sucked dry and transferred to a 2000 mL round bottom flask. 250 ml of 50% NaOH and 0.3 g of sodium borohydride were added, mechanical propeller stirring (250 rpm) was applied and the flask was immersed into a water bath at 50° C. After 30 minutes 92 mL of AGE was added. The reaction progressed for 18 h. The gel was washed 1×GV with distilled water, 5×GV with ethanol and then 8×GV with distilled water. Titration gave an allyl level of 288 μmol/mL of resin.

### a.2) Partial Activation (Outer Part of Bead)

600 g (mL) of allylated cross-linked agarose base matrix, was transferred and drained into a 4 L round bottomed flask together with 3 L of distilled water. Mechanical stirring was applied (250 rpm). A solution of 3.375 mL bromine in 400 mL of water was prepared. The bromine solution (equivalent to the allyls in a 7 μm shell of the 95.8 μm bead) was added through a drop funnel during approximately 15 minutes at a stirring speed of 300 rpm. After 20 minutes the gel was washed with 10×1GV with distilled water. Titration of remaining allyl groups indicated a shell (i.e., an outer part of the bead; a layer surrounding the core of the bead) of 4.5 μm.

### a.3) S-Shell Coupling

619 g (mL) of shell-activated gel from the previous step was transferred to a 2 L round bottom flask. 124 mL of distilled water and 148.5 g of sodium sulfite were added. Mechanical propeller stirring was applied and the solution was left for 30 min at 250 rpm. pH was adjusted to 12.5 by adding 50% NaOH and the flask was immersed into a water bath at 50° C. The reaction progressed for 21 h. The gel was washed 10×GV with distilled water. Titration of the sulfonic groups gave an ionic capacity (i.e., ligand density) of 73 μmol/mL.

### a.4) Vinyl Pyrrolidone Core Polymerization

25.4 g of the S-functionalized partially allylated material from the previous step was transferred to a glass filter. The resin was washed with 7×2 GV of distilled water followed by 3×1.5 GV of 1 M Na2SO4 and sucked dry. The dry gel was transferred to a 100 mL Duran flask and 1 M Na2SO4 solution was added to a total weight of 36.6 g. followed by the addition of 4.3 mL of vinyl pyrrolidone and 0.089 g of ADBA. Nitrogen gas was bubbled through the reaction solution with a Pasteur pipette for 20 min. The Duran flask was immersed in a glycerol bath with the temperature set at 45° C. and the stirring rate at 250 rpm. After 17 hours, 32.5 g of distilled water was added, and the suspension was allowed to stir for 30 min at the reaction temperature. The gel was transferred to a glass filter and was washed with 10 GV of distilled water, then with 5×1 GV ethanol and finally 10×1 GV water again. Dry weight analysis indicated an increase of weight of 126 mg/mL of resin (corresponding to the amount of polyvinylpyrrolidone (PVP) added in the core). The ionic capacity of the material was again measured to be 73 μmol/mL.

### Equipment and Samples

The sulfonate semi-solid resin prototype was packed in a Tricorn 10/150 column (Cytiva, Sweden) using water. The resin was packed at 0.8 mL/min until the resin was settled. After the resin has settled the resin was packed for >10 min at 3 mL/min. The top adaptor was adjusted to the top of the resin bed plus % turn of the top adaptor clockwise. The bed-height of the column was 14.6 cm and asymmetry of the column was tested by injecting 100 μL acetone (20 mg/mL) and a flow rate of 1.0 mL/min using water and an AKTA Avant 25 system (Cytiva, Sweden). The asymmetry was calculated to 1.27 (Spec. 0.8-1.8). The reference resin, SP Sepharose Fast Flow (SP FF), with same particle size distribution as the sulfonate semi-solid resin prototype was also packed in the same manner into a Tricorn 10/150 column with similar bed-height, 14.3 cm, as for the sulfonate semi-solid resin prototype. The asymmetry for the reference column (SP FF) was 1.48 (Spec. 0-8-1.8).

The columns were attached onto an AKTA Pure 25 system (Cytiva, Sweden). Each column was equilibrated with 3 column volumes (CV) A-buffer at 2 mL/min, 50 mM Na-acetate pH 5.0. 200 μL of a protein mixture containing monoclonal antibody (mAb) 17 mg/mL, Cytochrome C (CytC) 4 mg/mL and Lysozyme (Lys) 4 mg/mL at a flow rate of 1 mL/min. The column was washed with 2 CV A-buffer. The proteins were eluted using a linear gradient during 15 CV, 0-100% B, using 25 mM Na-phosphate +0.4 M NaCl pH 7.0 as B-buffer at 1 mL/min. Finally, the column was washed with 2 CV B-buffer and 2 CV A-buffer at 2 mL/min. Detection was at 280 nm.

### Results

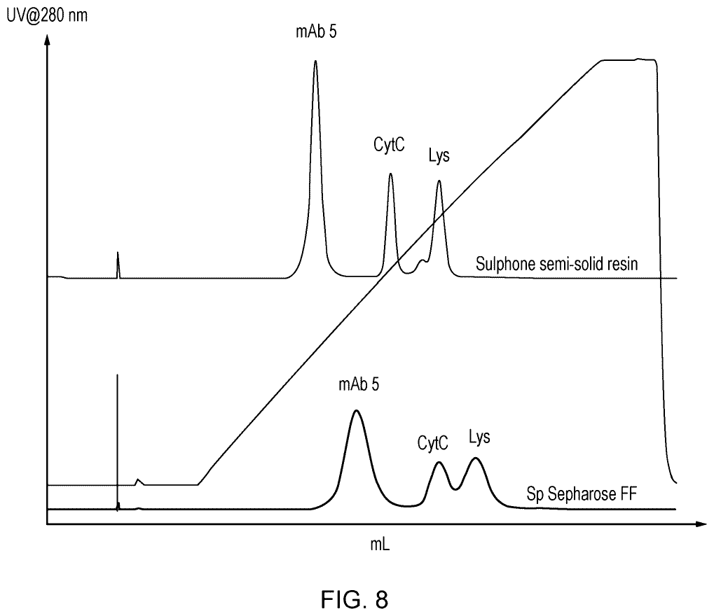

The peak-widths of the eluted proteins in FIG. 8 were determined and resolution, Rs, between mAb5-CytC and CytC-Lys peaks was evaluated, summarized in Table 2 below.

TABLE 2

| | Peak width | Peak width | Peak width | Rs | Rs |
|---|---|---|---|---|---|
| | Mab 5 | CytC | Lys | Mab | CytC- |
| Prototype | (mL) | (mL) | (mL) | 5-CytC | Lys |
| Sulfonate semi- | 3.6 | 2.7 | 3.5 | 5.9 | 4.0 |
| solid resin | | | | | |
| prototype | | | | | |
| SP FF reference | 7.3 | 9.2 | 11.2 | 3.1 | 1.6 |

### Conclusions

The sulfonate semi-solid resin gave narrower and more resolved chromatographic peaks than the reference SP Sepharose FF using a linear gradient, even if the particle size distribution was equal between the prototypes.

### Example 3: Separation of AAV9 Capsids Under Variable Conditions

Experimental designs for separation of fully packaged AAV9 capsids from empty AAV9 capsids and partially packaged AAV9 capsids are performed with equipment and samples as in Example 1 above, by use of weak cation exchange chromatography material on semi-solid beads as prepared in Example 1 item (a)+(b), with the following variations:

In terms of semi-solid bead preparation:
1) Reversing the order of method steps compared to Example 1 item (a)+(b), i.e., this variation comprises a method wherein a first step comprises preparing semi-solid beads, and a second step comprises functionalizing the semi-solid beads by coupling of ligands to the semi-solid beads, which ligands are for binding to target molecules. More particularly, the variation comprises a method for preparing chromatography particles according to the present disclosure, the method comprising a first step comprising preparing chromatography particles comprising a core and a layer surrounding the core, wherein the core has a first average pore diameter and the layer surrounding the core has a second average pore diameter, wherein the second average pore diameter is at least 1.5 times higher than the first average pore diameter, and a subsequent step comprising coupling of ligands for binding of target molecules to the semi-solid beads.

In terms of ligand density on semi-solid bead:
1) CM prototype on semi-solid beads of Example 1 item (b): Lower and higher densities of the same ligand.

In terms of ligand chemistry on semi-solid bead:
1) Weak cation exchange chromatography material:
   a. Dextran CM, i.e., carboxymethyl (CM) ligand (as in Example 1) coupled to dextran which is coupled to the support of the chromatography material;
   b. CM connected with an amide bond to the chromatography material;
   c. Poly CM or multiple CM bound to a short linker or scaffold, which is coupled to the support of the chromatography material;
2) Strong cation exchange chromatography material:
   a. Sulfonate (S) ligand (as in Example 2)

### Example 4: Separation of Target Molecules Under Variable Conditions

Experimental designs for separation of various target molecules are performed with equipment and samples as in Example 1 or Example 2 above, by use of chromatography material on semi-solid beads as prepared in Example 1 item (a)+(b) or Example 2 item (a), with the following variations:

In terms of ligand chemistry on semi-solid bead:
1) Strong anion exchange chromatography material:
   a. The ligand of the chromatography material Capto Q (Cytiva, Sweden), having about 100% quaternized amine groups;
   b. Dextran Capto Q ligand, i.e., Capto Q ligand coupled to dextran which is coupled to the support of the chromatography material;
   c. The ligand of the chromatography material Capto DEAE (Cytiva, Sweden), having a degree of quaternization of the amine groups of about 15%;
   d. Dextran Capto DEAE ligand, i.e., Capto DEAE ligand coupled to dextran which is coupled to the support of the chromatography material;
2) Strong cation exchange material:
   a. The ligand of the chromatography material Capto S ImpAct (Cytiva, Sweden), which comprises a sulfonate (S) group as the strong cation exchanger ligand;
   b. The ligand of the chromatography material Capto SP ImpRes (Cytiva, Sweden), which ligand comprises a sulphopropyl (SP) group;
3) Multimodal anion exchange chromatography material comprising hydrophobic groups:
   a. The ligand of the chromatography material Capto adhere ImpRes (Cytiva, Sweden), which comprises a N-benzyl-N-methyl ethanol amine ligand coupled to a support, wherein said support is linked to the nitrogen atom of the ligand through a linker.
4) Multimodal weak cation exchange chromatography material:
   a. The ligand of the chromatography material Capto MMC (Cytiva, Sweden), which ligand comprises a carboxylic group, and involves hydrogen bonding and hydrophobic interactions.
5) Hydrophobic interaction chromatography material:
   a. The ligand of the chromatography material phenyl Sepharose 6 Fast Flow (Cytiva, Sweden);
   b. The ligand of the chromatography material butyl Sepharose 4 Fast Flow (Cytiva, Sweden.

In terms of target molecule to be separated on semi-solid bead:
1) Adeno-associated virus capsids of serotype 8 (AAV8), 5 (AAV5), 1 (AAV1), 2 (AAV2), 4 (AAV4), 6 (AAV6), 7 (AAV7), or 10 (AAV10); to be separated on semi-solid bead comprising an anion exchange ligand as exemplified in Example 2 or Example 4 herein;
2) Monoclonal antibody variant, e.g., bispecific, fusion, or chimeric monoclonal antibody; to be separated on semi-solid bead comprising a ligand as exemplified in Example 2 and Example 4 herein;
3) Antibody fragment (including but not limited to antigen-binding fragments F(ab′)2, Fab, Fab′, and Fv; to be separated on semi-solid bead comprising a ligand as exemplified in Example 2 and Example 4 herein;
4) Oligonucleotide; to be separated on semi-solid bead comprising an anion exchange ligand, multimodal anion exchange ligand or hydrophobic interaction ligand as exemplified in Example 4 herein;
5) RNA; to be separated on semi-solid bead comprising an anion exchange ligand, multimodal anion exchange ligand or hydrophobic interaction ligand as exemplified in Example 4 herein;
6) DNA; to be separated on semi-solid bead comprising an anion exchange ligand, multimodal anion exchange ligand or hydrophobic interaction ligand as exemplified in Example 4 herein.

### Example 5: Determination of Pore Size Distributions and Relative Average Pore Diameters

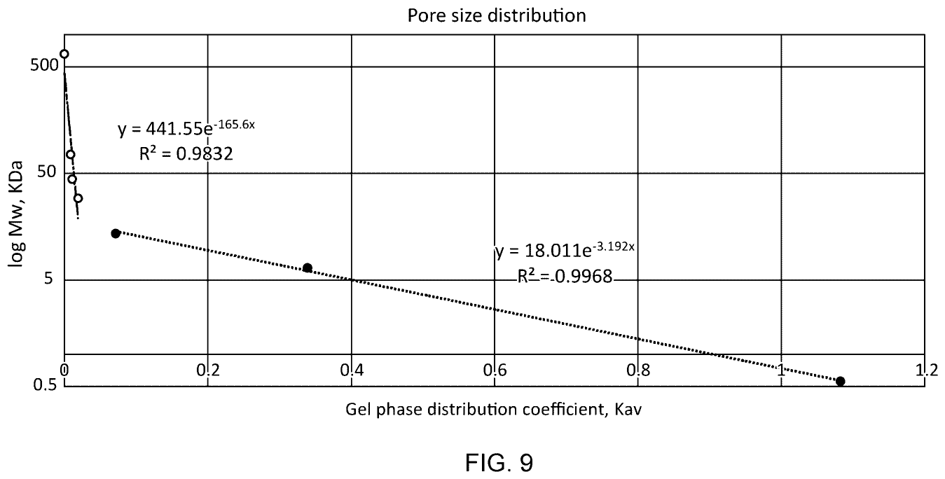

This example illustrates a method of verifying that the chromatography particles described herein have two different pore size distributions, one of the core and one of the outer layer, respectively. In summary, proteins and peptides of different sizes were injected and allowed to flow through a column packed with the chromatography beads produced as described herein. The retention volume for each polypeptide was determined and the distribution coefficient was calculated for each polypeptide. The distribution coefficient was plotted with the logarithmic molecular weight for each polypeptide (FIG. 9).

### Equipment and Materials

Chromatography resin (semi-solid beads) prepared essentially as described in Example 2a above was used. The particle size (d50v) of the agarose base matrix was 96.5 μm. Titration indicated an allyl level (C_{a11yl1}) after activation of 296.8 μmol/mL, and an allyl level (C_{allyl2}) following partial inactivation of 224.4 μmol/mL (cf. steps 2a.1 and 2a.2 above). The outer layer (shell) thickness, t₅, was calculated to be 4.3 μm using the formula

$${t}_{s} = {r}_{0} ( 1 - \sqrt[3]{\frac{{C}_{allyl 2}}{{C}_{allyl 1}}} )$$

in which r₀ is d50v/2, which corresponds to the radius of the bead, and C_{allyl1} and C_{allyl2} respectively represent the allyl level before and after the partial inactivation, as indicated above.

Furthermore, polypeptides as outlined in Table 3 were used.

TABLE 3

| | Molecular | |
|---|---|---|
| | weight | |
| Molecule | (MW) | Source |
| Thyroglobulin | 660 KDa | Sigma, art no T1001 |
| Conalbumin | 75 KDa | Cytiva, LMW kit art no |
| | | 28403841 lot 17640514 |
| Ovalbumin | 44 KDa | Cytiva, LMW kit art no |
| | | 28403841 lot 17640514 |
| Carbonic | 29 KDa | Cytiva, LMW kit art no |
| anhydrase | | 28403841 lot 17640514 |
| Ribonuclease | 13.7 KDa | Cytiva, LMW kit art no |
| A | | 28403841 lot 17640514 |
| A Protinin | 6.5 KDa | Cytiva, LMW kit art no |
| | | 28403841 lot 17640514 |
| Peptide | Average | Sigma, H2016-1VL Sigma |
| standard | Mw | lot 1003291564 |
| mix | 0.56 KDa | |

Mobile phase buffer: PBS (2 tablets; Medicago, art no 09-9400-00) and 58.4 g NaCl was dissolved in 2000 ml mQ water. The buffer contained 0.64 M NaCl, 0.0027 M KCl and 0.01 M Phosphate, pH 7.4.

### Procedure

The resin was packed in a Tricorn 10/300 column (Cytiva, Sweden) at a flow rate of 7 mL/min using 0.2 M NaCl (Merck) as packing eluent. The bed height was 29.3 cm with a total bed volume of 23.0 mL. The asymmetry was determined using an AKTA Pure 25 chromatography system (Cytiva, Sweden) injecting 100 μL 2% (w/v) acetone onto the column and using a flow-rate of 0.75 mL/min and UV detection at 280 nm. The asymmetry was found to be 0.9 which was approved. Generally, a column asymmetry value of between 0.8 and 1.8 is considered acceptable.

After asymmetry testing, the column was transferred and connected to a UHPLC system (Agilent 1260 Infinity). The proteins/peptides listed in Table 3 were weighed and diluted individually each to a final concentration of 3 mg/mL in 200 μL mobile phase buffer. 50 μL was injected individually and the molecules were allowed to migrate through the column at a flow rate of 0.8 mL/min. Detection was made using UV at 280 and 220 nm.

After chromatography the individual peaks of the chromatogram were integrated, and the retention time and retention volume were determined.

### Calculations and Results

Table 4 shows the calculations made for the column packed with the inventive resin. The void volume was determined using thyroglobulin, which has a molecular weight of 660 kDa.

The column volume was calculated by multiplying the bed height (29.3 cm) with the radius of the column (0.5 cm) raised to two and π.

Volume beads (Vb) was calculated by subtracting the column volume with the void volume.

Volume full particle (Vfp) was calculated by the formula: Vfp=4/3×π×(particle diameter/2)³

Volume layer (VI) was calculated by the formula: VI=Vfp−4/3×π×(particle diameter−(layer thickness×2)/2)³

TABLE 4
Calculated column properties

| Column volume: | 23.0 | mL |
|---|---|---|
| Flow rate: | 0.8 | mL/min |
| Void volume: | 8.36 | mL |
| Volume beads, Vb: | 14.65 | mL |
| Particle size: | 96.5 | μm |
| Layer thickness: | 4.3 | μm |
| Volume full particle, Vfp: | 470524 | μm³ |
| Volume layer, VI: | 60138 | μm³ |
| Layer volume of total particle (%): | 24.4% | |
| Volume layer in column: | 3.58 | mL |

Table 5 shows the retention volumes and the gel phase distribution coefficient (Kₐᵥ) for each polypeptide. Kₐᵥ was calculated by subtracting the retention volume (Vr) with the void volume. Vo, divided by the volume beads (Vb; 8.36 mL).

Kₐᵥ shell was calculated for the largest polypeptides by subtracting the retention volume (Vr) with the void volume, Vo, divided by the volume layer in the column (3.58 mL).

TABLE 5
Retention volumes and distribution coefficients

| | Molecular | Retention | Retention | | | |
|---|---|---|---|---|---|---|
| Molecule | weight (Da) | time (min) | volume, Vr (mL) | Vr − Vo | Kₐᵥ | Kₐᵥ shell |
| Thyroglobulin | 660 | 10.45 | 8.36 | 0 | 0 | 0 |
| (void marker Vo) | | | | | | |
| Conalbumin | 75 | 10.609 | 8.49 | 0.127 | 0.0087 | 0.0355 |
| Ovalbumin | 44 | 10.649 | 8.52 | 0.159 | 0.0109 | 0.0445 |
| Carbonic anhydrase | 29 | 10.8 | 8.64 | 0.28 | 0.0191 | 0.0782 |
| Ribonuclease A | 13.7 | 11.76 | 9.41 | 1.05 | 0.0715 | |
| A Protinin | 6.5 | 16.65 | 13.32 | 4.96 | 0.339 | |
| Peptide mix | 0.56 | 30.27 | 24.22 | 15.86 | 1.082 | |

FIG. 9 shows the log Mw for the polypeptides plotted against Kₐᵥ. Trend lines were created in Microsoft Excel with its linear equation, R>0.98 for both trend lines. It is clearly seen that the slope of the pore size distribution curves differ between the large biomolecules (29-660 kDa) and the smaller ones (0.6-14 kDa). From the differences in slopes it can be concluded that the resin bead has two pore size distributions. Since the volume of the porous outer layer was small compared to the volume of total beads, the large polypeptides were only able to migrate into the outer layer, not the core, and thus eluted early, generating a steep pore size distribution curve.

For the dense core of the beads, the exclusion limit (were the linear curve passes 0 on the y-axis) is 18 kDa and the fractionation range (Kₐᵥ 1 to 0) is then 0.7-18 kDa, based on the linear function in FIG. 9. For the outer layer, the slope of the pore size distribution curve indicates that it retards larger biomolecules in a linear relationship with the log molecular weight above the exclusion limit of 18 kDa for the dense core and up to 440 kDa.

### Conclusions

The average molecular weight for the linear fractionation range (0.7-18 kDa) of the core is 9 kDa, and for the fractionation range of the outer layer (18-440 kDa), the average molecular weight is around 200 KDa. This means that the average pore diameter of the outer layer is about 20 times larger than the average pore diameter of the core.

It is to be understood that the present disclosure is not restricted to the above-described exemplifying embodiments thereof and that several conceivable modifications of the present disclosure are possible within the scope of the following claims.

### REFERENCES

T. Edge, Chapter 4—Turbulent flow chromatography in bioanalysis (pp. 91-128), Handbook of Analytical Separations, Volume 4, pp. 1-425, 2003, Bioanalytical Separations, ISBN: 978-0-444-50658-0.
P. J. Marriott, Article on Gas chromatography—Principles (pp. 7-18), Encyclopedia of Analytical Science, Second Edition, 2005, ISBN 978-0-12-369397-6.
Weihong Qu et al, Scalable Downstream Strategies for Purification of Recombinant Adeno-Associated Virus Vectors in Light of the Properties, Current Pharmaceutical Biotechnology 2015 August; 16(8): 684-695.
Xiaotong Fu et al, Analytical Strategies for Quantification of Adeno-Associated Virus Empty Capsids to Support Process Development, Human gene therapy methods, 2019, 30(4): 144-152.
S Hjerten, Biochim Biophys Acta 79(2), 393-398,1964.
U.S. Pat. No. 6,602,990
R Arshady, Chimica e LIndustria 70(9), 70-75, 1988.
WO2006043896 A1
U.S. Pat. No. 6,428,707

1. A separation matrix comprising a plurality of chromatography particles, each chromatography particle comprising a core and a layer surrounding the core, wherein the core has a first average pore diameter and the layer surrounding the core has a second average pore diameter, wherein the second average pore diameter is at least 1.5 times higher than the first average pore diameter,
wherein the first average pore diameter excludes diffusion of a target molecule through the pores of the core and wherein the second average pore diameter at least partly permits diffusion of the target molecule through the pores of the layer surrounding the core.

2. The separation matrix according to claim 1, wherein the first average pore diameter excludes diffusion of a target molecule having a molecular weight of from about 50 kDa, such as about 140 kDa, such as about 2000 kDa.

3. The separation matrix according to claim 1, wherein the first average pore diameter excludes diffusion of a target molecule having a hydrodynamic diameter of from about 7 nm, such as about 10 nm, such as about 20 nm, such as about 25 nm.

4. The separation matrix according to claim 1, wherein the chromatography particle has a diameter of from about 20 μm to about 500 μm, such as from about 60 μm to about 120 μm, or such as from about 100 μm to about 400 μm.

5. The separation matrix according to claim 1, wherein the core has a diameter of from about 60% to about 97% of the diameter of the chromatography particle.

6. The separation matrix according to claim 1, wherein the layer surrounding the core has a diameter of from about 3% to about 40%, such as from about 6% to about 10% of the diameter of the chromatography particle.

7. The separation matrix according to claim 1, wherein the chromatography particle comprises a ligand for binding to the target molecule, wherein the ligand is located in the layer surrounding the core and optionally wherein the ligand is additionally located in the core.

8. The separation matrix according to claim 7, wherein the ligand is a cationic ligand, an anionic ligand, a hydrophobic interaction ligand, a multimodal ligand, or combinations thereof.

9. The separation matrix according to claim 7, wherein the ligand is connected to the surface of the chromatography particle via a linker or extender.

10. The separation matrix according to claim 1, wherein the chromatography particle is substantially spherical.

11. The separation matrix according to claim 1, wherein the chromatography particle is of natural or synthetic origin, preferably a polysaccharide, such as agarose.

12. The separation matrix according to claim 1, wherein the layer surrounding the core is a first layer and the chromatography particle further comprises a second layer surrounding the first layer,
wherein the second layer has a third average pore diameter which is different from the first average pore diameter and the second average pore diameter.

13. The separation matrix according to claim 1, wherein the core comprises a polymeric compound, which decreases the average pore diameter of the core compared to the average pore diameter of the layer(s) surrounding the core; optionally wherein the polymeric compound is a natural polymer, such as dextran, or a synthetic polymer.

14. A method for preparing a separation matrix according to claim 1, the method comprising preparing a plurality of chromatography particles, comprising:
(a) providing a chromatography particle having a homogeneous average pore diameter as starting material, wherein the homogeneous average pore diameter at least partly permits a target molecule to diffuse through the pores of the chromatography particle, throughout the entire structure or volume of the chromatography particle, and
(b) adding a polymeric compound inside a core of the chromatography particle provided in step (a), thereby decreasing the average pore diameter of the core compared to the homogeneous average pore diameter of the chromatography particle provided in (a), thereby obtaining a chromatography particle comprising a core and a layer surrounding the core, wherein the core has a first average pore diameter and the layer surrounding the core has a second average pore diameter, wherein the second average pore diameter corresponds to the homogeneous average core diameter of the chromatography particle provided in (a);
or:
(a′) providing a chromatography particle having a first average pore diameter, which excludes diffusion of a target molecule through the pores of the chromatography particle, throughout the entire structure or volume of the chromatography particle, and
(b′) embedding the chromatography particle provided in step (a′) in a surrounding layer having a second average pore diameter;
wherein the first average pore diameter and the second average pore diameter are different from each other, wherein the second average pore diameter is at least 1.5 times higher than the first average pore diameter,
wherein the first average pore diameter excludes diffusion of a target molecule through the pores of the core and wherein the second average pore diameter at least partly permits diffusion of the target molecule through the pores of the layer surrounding the core.

15. The method according to claim 14, wherein the adding of step (b) comprises:
(i) coupling of a polymeric molecule to the core of the particle; or
(ii) polymeric grafting, comprising adding monomers to the core of the particle, which monomers are allowed to polymerize inside the core, thereby creating a polymeric molecule inside the core;
thereby decreasing the average pore diameter of the core.

16. Use of the separation matrix according to claim 1 for preparative applications, such as for separating a target molecule from a cell culture harvest or impurities.

17. The use according to claim 16, wherein the target molecule is an adeno-associated virus capsid, a monoclonal antibody, an antigen-binding fragment of an antibody, a virus-like particle, a lentivirus capsid, an adenovirus capsid, or an influenza virus capsid.

18. A method of preparative chromatography for separating one or more target molecules from one or more other compounds in a liquid sample, which method comprises contacting a liquid sample comprising the target molecule(s) and compound(s) with a separation matrix according to claim 1, and which method comprises exposing the liquid sample to a pressure of <5 bar, such as <3 bar.

19. The method according to claim 18, comprising a step of clarification and/or chromatography preceding the step of contacting the liquid sample comprising the target molecule(s) and compound(s) with the separation matrix.

20. A method for separating adeno-associated virus capsids fully packaged with genetic material from adeno-associated virus capsids not fully packaged with genetic material, the method comprising the following steps:
a) adding a liquid sample comprising adeno-associated virus capsids to a weak cation exchange chromatography material,
wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
wherein the weak cation exchange chromatography material comprises:
i. a ligand for binding to the adeno-associated virus capsids, and
ii. a support, which comprises the separation matrix according to claim 1;
b) eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material;
optionally wherein the adeno-associated virus capsids are capsids of adeno-associated virus serotype 9 (AAV9) or a variant thereof.

21. The method according to claim 20, wherein the ligand of the weak cation exchange chromatography material is defined by the following Formula I: 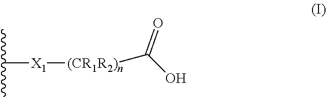
wherein
X₁ is selected from O, S, and NHCO;
n is an integer of from 1 to 4; and
each R₁ and R₂ is independently selected from hydrogen, C₁-C₃ alkyl, and OH.

22. The method according to claim 21, wherein X₁ is O and n is 1; or X₁ is S and n is 2; and each R₁ and R₂ is hydrogen.

23. The method according to claim 21, wherein the density of ligand is from about 30 to about 110 μmol, such as from about 40 to about 90 μmol, such as from about 50 to about 80 μmol, of ligand per ml of the weak cation exchange chromatography material.

24. The method according to claim 20, further comprising subjecting the eluate fractions comprising adeno-associated virus capsids fully packaged with genetic material, eluted in step (b) of claim 20, to one or more of the following steps:
c1) concentrating the adeno-associated virus capsids to a pharmaceutically relevant dose,
c2) replacing a buffer applied in step (b) of claim 20 with a pharmaceutically acceptable buffer, and/or
c3) sterilizing the eluate fractions comprising adeno-associated virus capsids,
thereby obtaining a pharmaceutical composition comprising adeno-associated virus capsids.

25. A composition comprising adeno-associated virus capsids obtained by performing the method of claim 20, in which composition the ratio of adeno-associated virus capsids fully packaged with genetic material to adeno-associated virus capsids not fully packaged with genetic material is at least 3:2, preferably at least 4:1.
